(19) United States
(12) Patent Application Publication
(10) Pub. No.: US 2026/0295219 A1
(43) Pub. Date: Oct. 1, 2026

(54) REPOSITIONING SHEATH FOR USE IN A PERCUTANEOUS CIRCULATORY SUPPORT SYSTEM

(71) Applicant: Boston Scientific Scimed, Inc., Maple Grove, MN (US)

(72) Inventors: Nicholas Lee Tassoni, Andover, MN (US); Daniel Frank Massimini, Brooklyn Park, MN (US); Judith Tiferes Wang, Shoreview, MN (US); Steffen Thomas Friedrich, Saxony (DE)

(73) Assignee: Boston Scientific Scimed, Inc., Maple Grove, MN (US)

(21) Appl. No.: 19/576,693

(22) Filed: Mar. 24, 2026

Related U.S. Application Data
(60) Provisional application No. 63/862,453, filed on Aug. 12, 2025.
(60) Provisional application No. 63/777,419, filed on Mar. 25, 2025.

(51) Int. Cl. A61M 25/06; A61M 25/00; A61M 39/02; A61M 60/13; A61M 60/865; A61M 60/90

(52) U.S. Cl. CPC A61M 25/0662; A61M 25/0097; A61M 39/0247; A61M 60/13; A61M 60/865; A61M 60/90; A61M 2039/0258; A61M 2205/583; A61M 2210/12

(57) ABSTRACT

A repositioning sheath for use in a percutaneous circulatory support system includes a body portion including a lumen, a sheath extending from the body portion and configured to fluidly engage a vasculature, and a locking portion configured to shift between unlocked and locked configurations secured to the body portion. The locking portion includes a plurality of beams extending longitudinally, a polymeric tube disposed inward of the beams, and a locking mechanism configured to engage the tube against an elongate shaft disposed within the tube in the locked configuration. A percutaneous circulatory support system includes a blood pump, a housing, an elongate shaft extending from the blood pump to the housing, and a repositioning sheath. A sterile sleeve extends from the housing and surrounds at least a portion of the shaft and is configured to releasably couple to the locking portion. The locking portion may be self-biased toward the locked configuration.

### CROSS REFERENCE TO RELATED APPLICATIONS

This application claims the benefit of U.S. Provisional Patent Application Serial No. 63/862,453, filed on August 12, 2025. This application also claims the benefit of U.S. Provisional Patent Application Serial No. 63/777,419, filed on March 25, 2025. The disclosures of which are incorporated herein by reference.

### TECHNICAL FIELD

The disclosure relates generally to medical devices and more particularly to sheath configurations for use in a percutaneous circulatory support system.

### BACKGROUND

In various medical procedures, a medical device, such as a percutaneous circulatory support device, is advanced through a vascular access sheath to a desired position within a patient’s body. The medical device may be maintained in the patient’s body for an extended period of time. Precise and adjustable positioning of the medical device and/or elements thereof during therapy may be desired to maintain optimal performance of the system. Existing systems may involve repositioning outside of a sterile field, which may compromise sterility and disrupt procedural workflow. It is desirable to provide controlled intra-procedural positioning and/or positional adjustability while preserving a sealed and sterile interface with the patient.

A wide variety of medical devices and systems have been developed for medical use. These devices are manufactured by any one of a variety of different manufacturing methods and may be used according to any one of a variety of methods. There is an ongoing need to provide alternative medical devices as well as alternative methods for manufacturing and/or using medical devices.

### SUMMARY

In one example, a repositioning sheath for use in a percutaneous circulatory support system may comprise a body portion including a lumen extending therethrough, a sheath extending distally from the body portion, the sheath including a lumen in communication with the lumen of the body portion, wherein the sheath is configured to fluidly engage with a vasculature of the patient, and a locking portion secured to the body portion, wherein the locking portion is configured to shift between an unlocked configuration and a locked configuration. The locking portion may include a plurality of beams extending longitudinally from the body portion, a polymeric tube disposed radially inward of the plurality of beams, the polymeric tube including a lumen in communication with the lumen of the body portion, and a locking mechanism configured to selectively engage the polymeric tube against an elongate shaft disposed within the lumen of the polymeric tube in the locked configuration.

In addition, or alternatively, to any example disclosed herein, the locking mechanism includes at least one ramp element extending radially outward from each beam of the plurality of beams, and a rotatable member disposed radially outward of the plurality of beams. The rotatable member is configured to engage the at least one ramp element extending radially outward from each beam of the plurality of beams to shift the locking portion between the unlocked configuration and the locked configuration.

In addition, or alternatively, to any example disclosed herein, the plurality of beams are circumferentially spaced apart from each other.

In addition, or alternatively, to any example disclosed herein, the locking portion is configured to prevent relative translation of the elongate shaft through the lumen of the polymeric tube in the locked configuration.

In addition, or alternatively, to any example disclosed herein, the locking portion is configured to permit relative translation of the elongate shaft through the lumen of the polymeric tube in the unlocked configuration.

In addition, or alternatively, to any example disclosed herein, the body portion includes a pointer configured to cooperate with the rotatable member to visually communicate whether the locking portion is in the unlocked configuration or the locked configuration.

In addition, or alternatively, to any example disclosed herein, the rotatable member includes a locked indicator disposed on an outer surface of the rotatable member and an unlocked indicator disposed on the outer surface of the rotatable member.

In addition, or alternatively, to any example disclosed herein, the locking portion includes at least one first stop element, wherein the at least one first stop element is configured to engage at least one second stop element formed in the rotatable member in the unlocked configuration and wherein the at least one first stop element is configured to engage at least one third stop element formed in the rotatable member in the locked configuration.

In addition, or alternatively, to any example disclosed herein, and in a second example, a percutaneous circulatory support system may comprise a blood pump, a housing, an elongate shaft extending from the blood pump to the housing, and a repositioning sheath. The repositioning sheath may comprise a body portion including a lumen extending therethrough, a sheath extending distally from the body portion, the sheath including a lumen in communication with the lumen of the body portion, wherein the sheath is configured to fluidly engage with a vasculature of the patient, and a locking portion secured to the body portion, wherein the locking portion is configured to shift between an unlocked configuration and a locked configuration. A sterile sleeve may extend from the housing and surrounds at least a portion of the elongate shaft. The sterile sleeve may be configured to releasably couple to the locking portion of the repositioning sheath.

In addition, or alternatively, to any example disclosed herein, the locking portion of the repositioning sheath includes a proximal attachment structure configured to releasably couple a distal hub of the sterile sleeve to the locking portion of the repositioning sheath.

In addition, or alternatively, to any example disclosed herein, the proximal attachment structure includes a pair of tabs biased radially apart from each other.

In addition, or alternatively, to any example disclosed herein, each tab of the pair of tabs comprises a ledge configured to engage with the distal hub of the sterile sleeve to couple the distal hub of the sterile sleeve to the locking portion of the repositioning sheath.

In addition, or alternatively, to any example disclosed herein, the pair of tabs is configured to be squeezed radially toward each other to release the distal hub of the sterile sleeve from the locking portion of the repositioning sheath.

In addition, or alternatively, to any example disclosed herein, the distal hub of the sterile sleeve is configured to rotate relative to the proximal attachment structure of the locking portion of the repositioning sheath.

In addition, or alternatively, to any example disclosed herein, the locking portion of the repositioning sheath includes a plurality of beams extending longitudinally from the body portion, a polymeric tube disposed radially inward of the plurality of beams, the polymeric tube including a lumen in communication with the lumen of the body portion, and a locking mechanism configured to selectively engage the polymeric tube against the elongate shaft when the elongate shaft is disposed within the lumen of the polymeric tube in the locked configuration.

In addition, or alternatively, to any example disclosed herein, the proximal attachment structure is fixedly attached to the plurality of beams.

In addition, or alternatively, to any example disclosed herein, the locking mechanism is configured to permit the elongate shaft to slide therethrough when the elongate shaft is disposed therein in the unlocked configuration and the locking mechanism is configured to prevent the elongate shaft from sliding therethrough when the elongate shaft is disposed therein in the locked configuration.

In addition, or alternatively, to any example disclosed herein, and in a third example, a repositioning sheath for use in a percutaneous circulatory support system may comprise a body portion including a lumen extending therethrough, a sheath extending distally from the body portion, the sheath including a lumen in communication with the lumen of the body portion, wherein the sheath is configured to fluidly engage with a vasculature of the patient, and a locking portion secured to the body portion, wherein the locking portion is configured to shift between an unlocked configuration and a locked configuration. The locking portion may include a polymeric tube including a lumen in communication with the lumen of the body portion, wherein the lumen of the polymeric tube is configured to receive an elongate shaft therein, and a locking mechanism configured to squeeze the polymeric tube against the elongate shaft when the elongate shaft is disposed within the lumen of the polymeric tube in the locked configuration. The locking portion may be self-biased toward the locked configuration.

In addition, or alternatively, to any example disclosed herein, the locking mechanism includes a first button structure, a second button structure disposed opposite the first button relative to a central longitudinal axis of the polymeric tube, wherein the second button structure is slidably engaged with the first button structure, a first spring configured to bias a user-engageable portion of the first button structure away from the central longitudinal axis of the polymeric tube, and a second spring configured to bias a user-engageable portion of the second button structure away from the central longitudinal axis of the polymeric tube.

In addition, or alternatively, to any example disclosed herein, the first button structure and the second button structure cooperate to shift the locking portion from the locked configuration to the unlocked configuration such that only one of the first button structure and the second button structure cannot shift the locking portion from the locked configuration to the unlocked configuration on its own.

The above summary of some embodiments, aspects, and/or examples is not intended to describe each disclosed embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIG. 1 schematically illustrates selected aspects of a percutaneous circulatory support device including a percutaneous blood pump;

FIG. 2 schematically illustrates selected aspects of a percutaneous circulatory support system including a repositioning sheath in an unlocked configuration;

FIG. 2A is a partial cross-sectional view taken through the line 2A-2A in FIG. 2;

FIG. 3 is a partial cutaway and partial cross-sectional view schematically illustrating selected aspects of the percutaneous circulatory support system of FIG. 2 including the repositioning sheath in the unlocked configuration with a sterile sleeve coupled thereto;

FIG. 4 is a partial cutaway and partial cross-sectional view schematically illustrating selected aspects of the percutaneous circulatory support system of FIGS. 2-3 including the repositioning sheath in the unlocked configuration with the sterile sleeve being released therefrom;

FIG. 5 illustrates selected aspects of a rotatable member of the repositioning sheath of FIGS. 2-4;

FIG. 6 schematically illustrates selected aspects of the percutaneous circulatory support system of FIGS. 2-4 including the repositioning sheath in a locked configuration;

FIG. 6A is a partial cross-sectional view taken through the line 6A-6A in FIG. 6;

FIG. 7 is a partial cutaway view schematically illustrating selected aspects of the percutaneous circulatory support system of FIG. 6 including the repositioning sheath in the locked configuration;

FIG. 8 schematically illustrates selected aspects of a repositioning sheath including a viewing window;

FIG. 9 schematically illustrates selected aspects of a repositioning sheath including a viewing tube;

FIG. 10 schematically illustrates selected aspects of a percutaneous circulatory support system including a repositioning sheath in a locked configuration;

FIG. 11 is a partial cutaway view schematically illustrating selected aspects of the percutaneous circulatory support system of FIG. 10 including the repositioning sheath in the locked configuration;

FIG. 12 is a partial cross-sectional view taken through the line 12-12 in FIG. 11;

FIG. 13 schematically illustrates selected aspects of the percutaneous circulatory support system of FIGS. 10-12 including the repositioning sheath in an unlocked configuration;

FIG. 14 is a partial cutaway view schematically illustrating selected aspects of the percutaneous circulatory support system of FIG. 13 including the repositioning sheath in the unlocked configuration;

FIG. 15 is a partial cross-sectional view taken through the line 15-15 in FIG. 14;

FIG. 16 is a partial cutaway view schematically illustrating selected aspects of the percutaneous circulatory support system of FIG. 10 in the locked configuration;

FIG. 17 is a partial cross-sectional view taken through the line 17-17 in FIG. 16; and

FIG. 18 is a partial cutaway view schematically illustrating selected aspects of an alternative configuration of the percutaneous circulatory support system of FIGS. 10-11 in the locked configuration.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale and/or which may include changes of scale therein, wherein like reference numerals indicate like elements throughout the disclosure. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed. Additionally, the term “substantially”, when used in reference to two dimensions being substantially equal or the same, shall generally refer to a difference of less than or equal to 5%.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together unless such discrete structures or elements are no longer distinguishable from one another.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

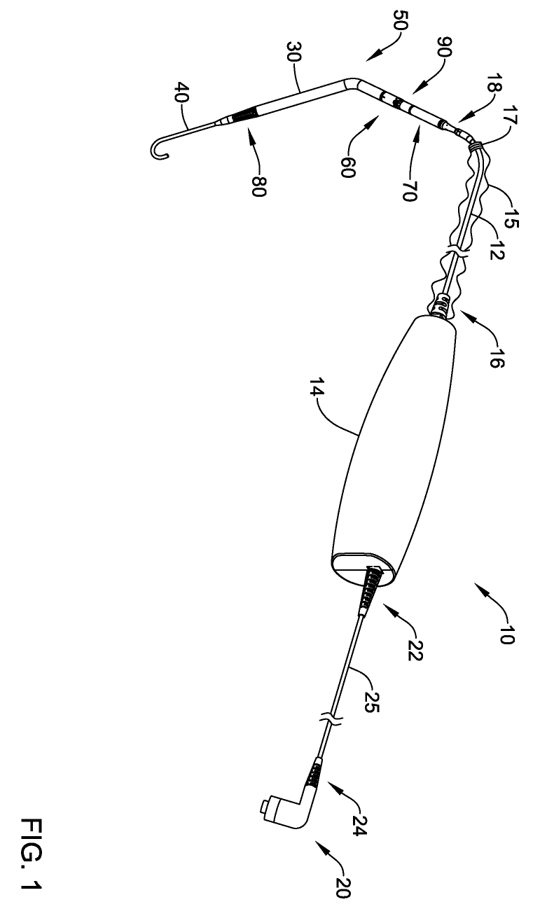

FIG. 1 illustrates a perspective view of a catheter 10 (e.g., a percutaneous circulatory support device and/or other suitable catheter) including a percutaneous blood pump 50 located at a distal end region thereof. Although not required, the blood pump 50 may be configured to pump blood from a ventricle of a heart of a patient to a vasculature of the patient. As such, the blood pump 50 and/or at a least a portion of the blood pump 50 may be positionable within the ventricle of the heart of the patient.

The blood pump 50 may generally include a flexible cannula 30, an impeller housing 60, and a motor housing 70. In some embodiments, the flexible cannula 30, the impeller housing 60, and/or the motor housing 70 may be integrally and/or monolithically constructed. In other instances, the flexible cannula 30, the impeller housing 60, and/or the motor housing 70 may be separate components. The impeller housing 60 comprises and/or carries an impeller assembly (not shown) therein. The impeller assembly may include an impeller secured to an impeller shaft that rotates relative to the impeller housing 60 to drive blood through the blood pump 50. In some embodiments, the impeller shaft and the impeller of the impeller assembly may be integrally and/or monolithically formed. In other embodiments, the impeller shaft and the impeller may be separate components.

Rotation of the impeller causes blood to flow from a blood inlet 80 of the blood pump 50, such as proximate and/or at a distal end of the flexible cannula 30, through the flexible cannula 30 and the impeller housing 60, and out of a blood outlet 90 proximal of the impeller, such as through a sidewall of the impeller housing 60. In some instances, the blood inlet 80 may include a plurality of blood inlet windows arranged around a circumference of the flexible cannula 30. In some instances, the blood outlet 90 may include a plurality of blood outflow windows arranged around a circumference of the impeller housing 60. In other embodiments, the blood inlet 80 and/or the blood outlet 90 may be formed on other portions of the blood pump 50.

The catheter 10 (e.g., the percutaneous circulatory support device) and/or a percutaneous circulatory support system may comprise a housing 14 (e.g., a housing of a handle, a control module, a junction box, etc.) positionable outside of the patient. In some embodiments, the catheter 10 (e.g., the percutaneous circulatory support device) may comprise an elongate shaft 12 (e.g., an elongate tube with one or more elongate members extending therein) coupled with the blood pump 50. The elongate shaft 12 may be disposed proximal of and/or may extend proximally from the blood pump 50 toward and/or to the housing 14. In some embodiments, the catheter 10 (e.g., the percutaneous circulatory support device) may comprise a distal tip 40 disposed distal of and/or extending distally from the blood pump 50 and/or the flexible cannula 30.

In some embodiments, a proximal end 16 of the elongate shaft 12 may be coupled to the housing 14 and a distal end 18 of the elongate shaft 12 may be coupled to the blood pump 50. In some embodiments, the proximal end 16 of the elongate shaft 12 may be fixedly attached to the housing 14 and/or the distal end 18 of the elongate shaft 12 may be fixedly attached to the blood pump 50. Other configurations are also contemplated.

The catheter 10 (e.g., the percutaneous circulatory support device) and/or a percutaneous circulatory support system may comprise a cable 25 extending between the housing 14 and a connector 20. In some embodiments, the cable 25 may have a first end and/or a distal end 22 connected to the housing 14, and a second end and/or a proximal end 24 connected to the connector 20. In some embodiments, the first end and/or the distal end 22 of the cable 25 may be attached directly to the housing 14. In some embodiments, the first end and/or the distal end 22 of the cable 25 may be fixedly attached to the housing 14.

The connector 20 may be configured to be connected to a controller (not shown) for controlling the blood pump 50, providing electrical power and/or control signals to the blood pump 50, etc. The cable 25 may include one or more of, an electrical pathway for powering a motor within the motor housing 70, an electrical pathway for powering one or more sensors associated with the blood pump 50, an electrical pathway for transmitting signals from one or more sensors associated with the blood pump 50, a fiber optic filament for transmitting signals from one or more sensors associated with the blood pump 50, and/or other communication conduits. In some instances, the catheter 10 (e.g., the percutaneous circulatory support device) and/or a percutaneous circulatory support system may include additional cables extending from the housing 14 configured to be connectable to the controller for sending and/or receiving signals, such as from one or more sensors during operation of the blood pump 50.

In some embodiments, when placing the blood pump 50 in a subject (e.g., the patient), such as within the ventricle of the heart of the patient, the blood pump 50 may be inserted through an introducer sheath inserted into a vascular access site of the subject. In some cases, the introducer sheath may be a large bore sheath configured to receive and facilitate the passage of the blood pump 50 and the elongate shaft 12 through a lumen thereof and into a blood vessel (e.g., a femoral artery, etc.) and/or the vasculature of the subject (e.g., the patient).

In some embodiments, the catheter 10 (e.g., the percutaneous circulatory support device) and/or the percutaneous circulatory support system may comprise a sterile sleeve 15 secured to and extending away from the housing 14. In at least some embodiments, the sterile sleeve 15 may extend distally from the housing 14. In some embodiments, the sterile sleeve 15 may be fixedly secured at and/or to a distal end of the housing 14. In some embodiments, the sterile sleeve 15 may include a distal hub 17 disposed at a distal end of the sterile sleeve 15.

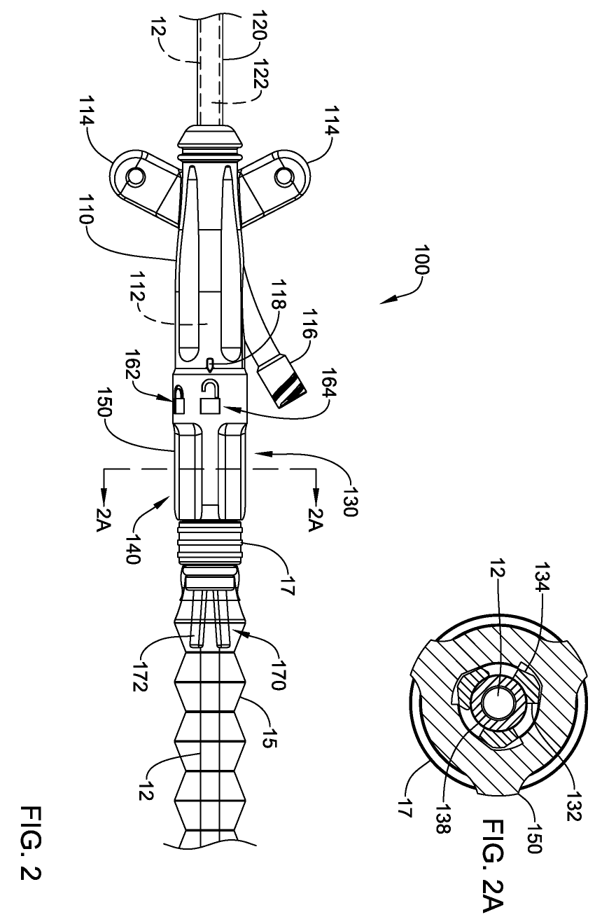

In some embodiments, the elongate shaft 12 of the catheter 10 (e.g., the percutaneous circulatory support device) may be disposed within the sterile sleeve 15 during use. In some embodiments, the sterile sleeve 15 may surround at least a portion of the elongate shaft 12 to maintain a sterile field around the elongate shaft 12 during use of the catheter 10 (e.g., the percutaneous circulatory support device) and/or the percutaneous circulatory support system (e.g., during a medical procedure). In some embodiments, the sterile sleeve 15 and/or the distal hub 17 may be configured to releasably couple to a repositioning sheath (e.g., ref. 100; FIG. 2) as discussed herein.

In some embodiments, the sterile sleeve 15 may be flexible and/or axially collapsible and/or axially expandable to a desired length. In some embodiments, the sterile sleeve 15 may be configured to have bunched locations (e.g., pleats, folds, wrinkles, etc.) depending on the length of the elongate shaft 12 and/or at or along bends formed in the elongate shaft 12. A bunched location may be a location along the sterile sleeve 15 where excess portions of the sterile sleeve 15 may be gathered or grouped together. Accordingly, in some embodiments, the bunched locations may allow for the length of the sterile sleeve 15 to be adjusted depending on the placement of the catheter 10 (e.g., the percutaneous circulatory support device) and/or the housing 14 during a medical procedure. In some embodiments, the sterile sleeve 15 may have a variable length or an adjustable length. In at least some embodiments, the elongate shaft 12 of the catheter 10 (e.g., the percutaneous circulatory support device) may be slidably disposable within the sterile sleeve 15 and/or the distal hub 17. In some embodiments, a length of the portion of the elongate shaft 12 of the catheter 10 (e.g., the percutaneous circulatory support device) disposed within the sterile sleeve 15 may be variable and/or adjustable.

The sterile sleeve 15, or portions thereof, may be formed from any suitable materials. Example suitable materials for forming the sterile sleeve 15 include, but are not limited to, polyamide, polyurethane, thermoplastic polyurethane (TPU), polyethylene (PE), polyethylene terephthalate (PET), polypropylene (PP), ethylene vinyl acetate (EVA), polytetrafluoroethylene (PTFE), fluorinated ethylene propylene (FEP), polyvinyl chloride (PVC), polyether block amides (PEBA), and/or other suitable materials. Some other suitable but non-limiting materials for the sterile sleeve 15, for example polymeric materials, are described below. In some embodiments, the sterile sleeve 15 may be substantially transparent and/or clear, such that the elongate shaft 12 of the catheter 10 (e.g., the percutaneous circulatory support device) is visible within the sterile sleeve 15, as shown in the figures. In some alternative embodiments, the sterile sleeve 15 may be translucent. In yet other alternative embodiments, the sterile sleeve 15 may be substantially opaque.

FIGS. 2-7 schematically illustrate selected aspects of a percutaneous circulatory support system including the catheter 10 (e.g., the percutaneous circulatory support device) positioned within a repositioning sheath 100 (e.g., a vascular access sheath, an introducer sheath, etc.) for use in the percutaneous circulatory support system. In some embodiments, the repositioning sheath 100 may be used for facilitating passage of various medical devices, such as the elongate shaft 12, the blood pump 50, etc., through a vascular access site of the subject (e.g., the patient) and into a blood vessel and/or the vasculature of the subject (e.g., the patient). In some embodiments, the repositioning sheath 100 may be configured to extend into the blood vessel and/or the vasculature of the subject (e.g., the patient) and allow the elongate shaft 12, the blood pump 50, and/or other suitable medical devices to pass therethrough. In at least some embodiments, during use, the elongate shaft 12 of the catheter 10 (e.g., the percutaneous circulatory support device) may be positionable within the repositioning sheath 100.

In some embodiments, the repositioning sheath 100 may comprise a body portion 110 including a lumen 112 extending therethrough and at least one wing 114 extending laterally from the body portion 110. The at least one wing 114 may be configured to be secured to the subject (e.g., the patient). In one non-limiting example, the at least one wing 114 may be sutured to skin of the subject (e.g., the patient). Other configurations are also contemplated. In some embodiments, the body portion 110 may include a hemostasis valve (not shown) disposed therein. The hemostasis valve may be configured to engage with the elongate shaft 12 of the catheter 10 (e.g., the percutaneous circulatory support device) to prevent and/or minimize blood leakage out of the repositioning sheath 100 during use. In at least some embodiments, the body portion 110 may include a side port 116 in fluid communication with the lumen 112 of the body portion 110. In some embodiments, the side port 116 may be fluidly coupled to a section of tubing (not shown), which may include a valve and/or a fluid port, in fluid communication with a source of fluid (not shown). In some embodiments, the side port 116 may be a guidewire port and/or may be configured to slidably receive a guidewire therein.

In some embodiments, the repositioning sheath 100 may comprise a sheath 120 extending distally from the body portion 110. The sheath 120 may include a lumen 122 in communication with the lumen 112 of the body portion 110. In at least some embodiments, the sheath 120 may be configured to fluidly engage with the blood vessel and/or the vasculature of the subject (e.g., the patient). In some embodiments, the sheath 120 may be fixedly attached to the body portion 110. In some embodiments, the sheath 120 may be integrally and/or monolithically formed with the body portion 110. In some alternative embodiments, the sheath 120 may be removably attached to the body portion 110.

In some embodiments, the sheath 120 may be configured to directly access and/or to extend directly into the blood vessel and/or the vasculature of the subject (e.g., the patient) via an arterial access site (e.g., a femoral access site, etc.). As such, in some embodiments, the sheath 120 may function as a vascular access sheath. In some embodiments, the blood pump 50 (e.g., FIG. 1) may be delivered to the heart of the patient via an arterial access site. In one non-limiting example, the blood pump 50 may be delivered to the heart of the patient via a femoral access site. However, utilizing a femoral access site may limit the mobility of the patient as movement of the elongate shaft 12 (extending out of a femoral access site) as the patient moves may result in inadvertent movement of the blood pump 50 in the heart of the patient. As such, to allow the patient to be ambulatory with the blood pump 50 operating in the heart of the patient, it may be advantageous to utilize an access site closer to the heart of the patient and/or not at a limb of the patient that moves relative to a core (e.g., relative to a chest) of the patient as the patient becomes ambulatory. In some examples, the axillary artery or subscapular artery may have a sufficient diameter to receive the blood pump and is close enough to the heart of the patient (e.g., located at the chest of the patient) to mitigate a likelihood of the elongate shaft 12 moving in a manner that would inadvertently change the position of the blood pump 50 in the heart of the patient as the patient becomes ambulatory.

In some alternative embodiments, the sheath 120 may be configured to fluidly couple with and/or be inserted into a graft (e.g., a port, an access tube, etc.) attached to, installed in, and/or fluidly coupled to the axillary artery or the subscapular artery. The graft may be attached to, installed in, and/or fluidly coupled to the axillary artery and/or the subscapular artery using a surgical technique or a percutaneous approach.

Using a surgical technique, the graft may be attached to the axillary artery or the subscapular artery and the blood pump 50 may be delivered to the heart of the patient via the axillary artery and/or the subscapular artery by inserting the blood pump 50 into the graft and advancing the blood pump 50 through the axillary artery and/or the subscapular artery to the heart. Use of the graft may facilitate providing an access site for inserting the blood pump 50 to the heart of the patient and through which the elongate shaft 12 may extend during operation of the blood pump 50, which may mitigate bleeding from the blood vessel and/or the vasculature and mitigate a likelihood of infection. Further, maintaining the graft at the access site may facilitate removal of the blood pump 50 from the patient after a period of time. Delivering the blood pump 50 to the heart of the patient via the axillary artery, the subscapular artery, and/or other suitable artery proximate the heart of the patient (e.g., at an upper extremity access site) may facilitate supporting a patient utilizing the blood pump 50 in an ambulatory state for an extended period of time (e.g., up to two weeks and/or beyond two weeks).

Utilizing a percutaneous approach for placing the graft may reduce bleeding and/or mitigate infection risks at the arterial access site for inserting the blood pump 50 into the vasculature of the patient. In some examples, a vascular access device with an elongate tubular frame and a graft or tubular extension extending from the elongate tubular frame may be inserted into an arterial system of the patient (e.g., via a radial artery or other suitable arterial access site) and delivered to a target location in the axillary artery or other suitable target location. Once the vascular access device is delivered to the target location, a cut or incision through the skin of the patient and the wall of the axillary artery of the patient may be made and the graft or tubular extension may be extended out through the wall of the axillary artery and out of the cut or incision through the skin of the patient. The blood pump 50 may then be delivered to the heart of the patient via the graft or tubular extension accessed in a subscapular region, for example, providing access to the axillary artery of the patient.

In some embodiments, a length of the sheath 120 may be related to and/or dependent upon how the sheath 120 is intended to engage with the vasculature of the patient. For example, when the sheath 120 is intended to be directly inserted into the vasculature of the patient and/or function as a vascular access sheath, the sheath 120 may be longer than if the sheath 120 is intended to be fluidly coupled with a graft in fluid communication with the axillary artery or the subscapular artery. In some embodiments, a form factor (e.g., shape, etc.) of the sheath 120 may change and/or be different to facilitate the different uses. In at least some embodiments, the sheath 120 may be formed from a polymeric material. Some suitable but non-limiting materials for the sheath 120, including polymeric materials, metallic materials, composites, and the like, are described below.

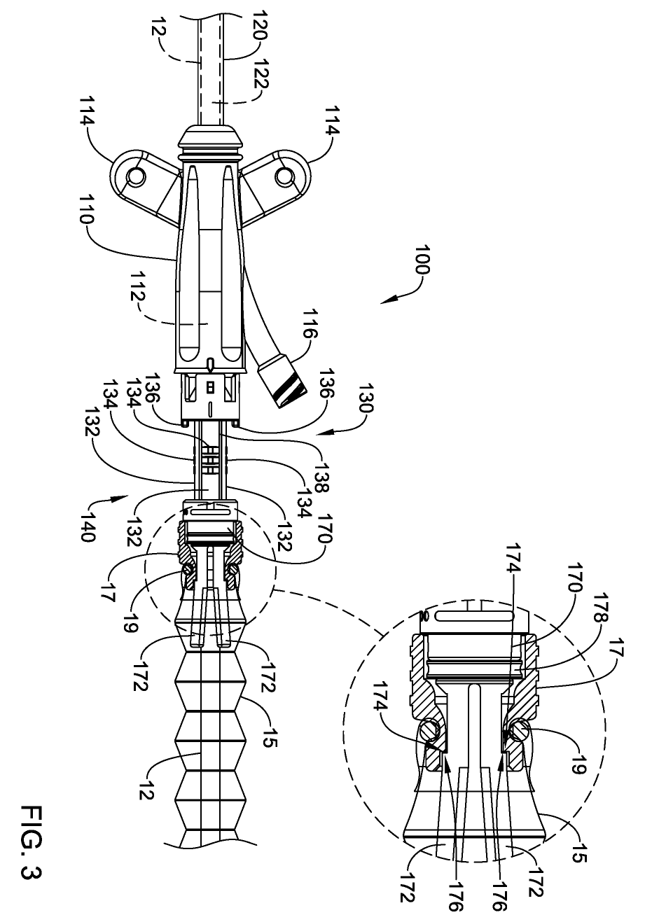

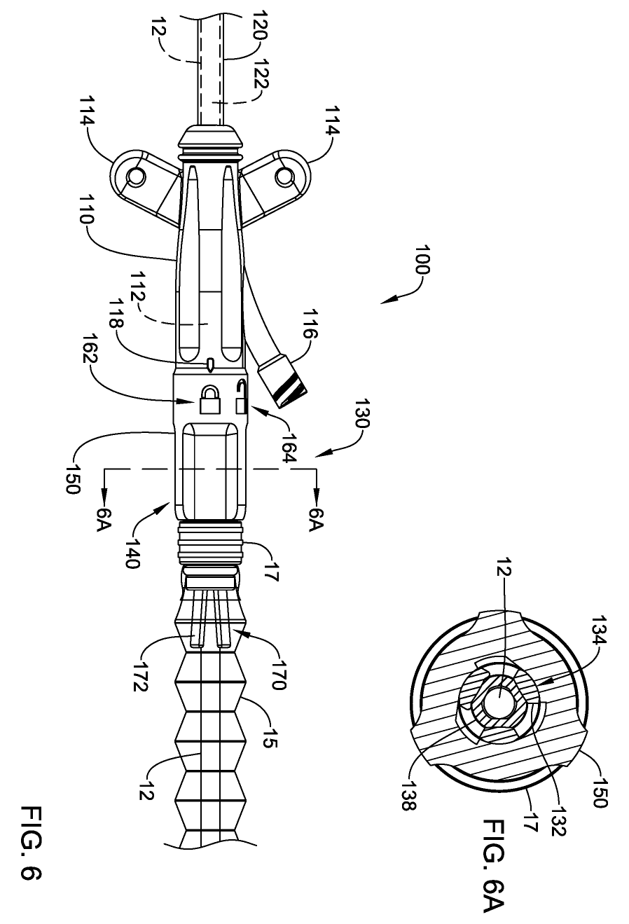

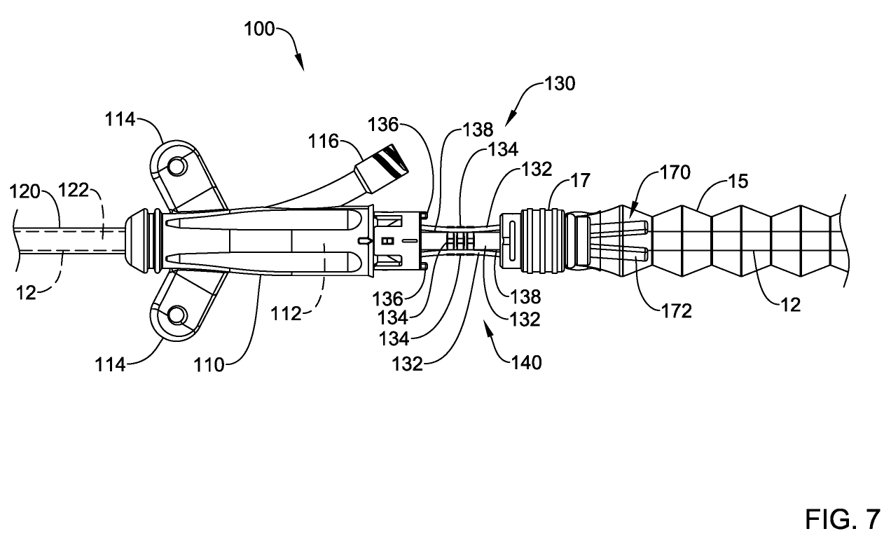

In some embodiments, the repositioning sheath 100 may comprise a locking portion 130 secured to the body portion 110. In some embodiments, the locking portion 130 may be fixedly attached to the body portion 110. In some embodiments, the locking portion 130 may be configured to shift between an unlocked configuration, as shown in FIGS. 2-3, and a locked configuration, as shown in FIGS. 6-7. To improve clarity and understanding, some elements of the repositioning sheath 100 are not shown in FIGS. 3 and 7.

In some embodiments, the locking portion 130 may include a plurality of beams 132 (e.g., FIG. 3) extending longitudinally from the body portion 110. In one example, the plurality of beams 132 may extend proximally from the body portion 110. In some embodiments, the locking portion 130 may include a lumen extending therethrough and/or radially inward from the plurality of beams 132. In some embodiments, the lumen of the locking portion 130 may be in communication with the lumen 112 of the body portion 110. In some embodiments, the plurality of beams 132 may be circumferentially spaced apart from each other. In some embodiments, each beam of the plurality of beams 132 may be circumferentially spaced apart from each circumferentially adjacent beam of the plurality of beams 132. In one non-limiting example, the plurality of beams 132 may comprise three beams. Other configurations and/or quantities of beams, for example, two beams, four beams, etc., are also contemplated.

In some embodiments, the locking portion 130 may include at least one first stop element 136 (e.g., FIG. 3). In some embodiments, the at least one first stop element 136 may include a plurality of first stop elements (e.g., two first stop elements, etc.). In some embodiments, the at least one first stop element 136 may extend longitudinally and/or may extend generally parallel to a central longitudinal axis of the locking portion 130. In some embodiments having a plurality of first stop elements (e.g., ref. 136), the plurality of first stop elements may be circumferentially spaced apart from each other around the central longitudinal axis of the locking portion 130.

In some embodiments, the locking portion 130 may include a polymeric tube 138 disposed therein and/or disposed within the lumen of the locking portion 130. The polymeric tube 138 may be disposed radially inward of the plurality of beams 132. The polymeric tube 138 may include a lumen in communication with the lumen 112 of the body portion 110. In some embodiments, the elongate shaft 12 of the catheter 10 (e.g., the percutaneous circulatory support device) may be slidably disposable within the lumen of the polymeric tube 138, the lumen 122 of the sheath 120, and the lumen 112 of the body portion 110.

In some embodiments, the locking portion 130 may include a locking mechanism 140 configured to selectively engage the polymeric tube 138 against the elongate shaft 12 of the catheter 10 (e.g., the percutaneous circulatory support device) disposed and/or positioned within the lumen of the polymeric tube 138 in the locked configuration (e.g., FIG. 6A). In some embodiments, the locking portion 130 may include a locking mechanism 140 configured to selectively squeeze the polymeric tube 138 against the elongate shaft 12 of the catheter 10 (e.g., the percutaneous circulatory support device) disposed and/or positioned within the lumen of the polymeric tube 138 in the locked configuration (e.g., FIG. 6A). In the unlocked configuration (e.g., FIG. 2A), the elongate shaft 12 of the catheter 10 (e.g., the percutaneous circulatory support device) may be slidably disposed within the lumen of the polymeric tube 138 and/or may be slidable through and/or within the locking portion 130 and/or the locking mechanism 140. In the locked configuration, the locking portion 130 and/or the locking mechanism 140 may be configured to prevent relative axial translation of the elongate shaft 12 of the catheter 10 (e.g., the percutaneous circulatory support device) within and/or through the repositioning sheath 100, the locking portion 130, the lumen of the polymeric tube 138, and/or the locking mechanism 140. In some embodiments, the locking portion 130 and/or the locking mechanism 140 may be configured to permit the elongate shaft 12 of the catheter 10 (e.g., the percutaneous circulatory support device) to slide therethrough in the unlocked configuration, and the locking mechanism 140 may be configured to prevent the elongate shaft 12 of the catheter 10 (e.g., the percutaneous circulatory support device) from sliding therethrough in the locked configuration.

In some embodiments, the polymeric tube 138 may be formed from a polymeric material such that when engaged with the elongate shaft 12 of the catheter 10 (e.g., the percutaneous circulatory support device), a frictional force between the polymeric tube 138 and the elongate shaft 12 of the catheter 10 (e.g., the percutaneous circulatory support device) is sufficient to prevent relative axial translation of the elongate shaft 12 of the catheter 10 (e.g., the percutaneous circulatory support device) within and/or through the repositioning sheath 100, the locking portion 130, the lumen of the polymeric tube 138, and/or the locking mechanism 140 during normal patient movement. In some examples, the frictional force may be at least 50 grams-force (490.33 milliNewton (mN)). In some examples, the frictional force may be at least 75 grams-force (735.50 mN). In one preferred but non-limiting example, the frictional force may be at least 100 grams-force (980.67 mN). In some examples, the frictional force may be at least 125 grams-force (1225.83 mN). In some examples, the frictional force may be at least 150 grams-force (1471.00 mN). In some examples, the frictional force may be at least 175 grams-force (1716.16 mN). In some examples, the frictional force may be at least 200 grams-force (1961.33 mN). In some examples, the frictional force may be between about 25 grams-force (245.17 mN) and about 250 grams-force (2451.66 mN). In some examples, the frictional force may be between about 50 grams-force (490.33 mN) and about 200 grams-force (1961.33 mN). In some examples, the frictional force may be between about 75 grams-force (735.50 mN) and about 150 grams-force (1471.00 mN). In some examples, the frictional force may be between about 100 grams-force (980.67 mN) and about 125 grams-force (1225.83 mN). Other examples and/or values are also contemplated. In one non-limiting example, the polymeric tube 138 may comprise and/or may be formed from silicone. Some other suitable but non-limiting polymeric materials for the polymeric tube 138 are described below.

In some embodiments, the locking mechanism 140 may include at least one ramp element 134 extending radially outward from each beam of the plurality of beams 132, as seen in FIGS. 2A and 3. In some embodiments, the locking mechanism 140 may include a rotatable member 150 disposed radially outward of and/or surrounding the plurality of beams 132 and the at least one ramp element 134 extending radially outward from each beam of the plurality of beams 132, as seen in FIGS. 2 and 2A. The rotatable member 150 is not shown in FIG. 3. In some embodiments, the rotatable member 150 may be configured to engage the at least one ramp element 134 extending radially outward from each beam of the plurality of beams 132 to shift the locking portion 130 and/or the locking mechanism 140 between the unlocked configuration (e.g., FIGS. 2-2A) and the locked configuration (e.g., FIGS. 6-6A).

As shown in FIGS. 2A and 3, in the unlocked configuration, the locking mechanism 140 and/or the rotatable member 150 may be configured to avoid deflection of the plurality of beams 132 in a radially inward direction. Accordingly, in the unlocked configuration, the plurality of beams 132 does not squeeze the polymeric tube 138 against the elongate shaft 12 disposed within the lumen of the polymeric tube 138.

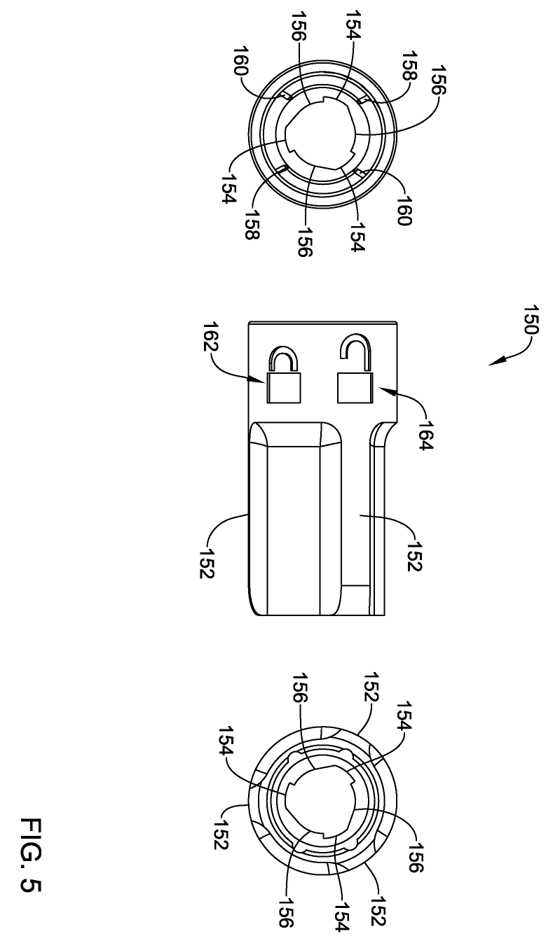

In some embodiments, the rotatable member 150 may be oriented substantially parallel to the central longitudinal axis of the polymeric tube 138. In some embodiments, the rotatable member 150 may be configured to rotate around an axis that is oriented parallel to the central longitudinal axis of the polymeric tube 138. In at least some embodiments, the rotatable member 150 may be configured to rotate around the central longitudinal axis of the polymeric tube 138. Some additional details regarding the rotatable member 150 are shown in FIG. 5 and described herein.

In some embodiments, the rotatable member 150 may include at least one raised rib 152 formed in an outer surface of the rotatable member 150, as seen in FIG. 5. In some embodiments, the at least one raised rib 152 may be configured to provide finger grip(s) for rotational engagement of the rotatable member 150 by a user’s hand (e.g., by a thumb and a finger, or by a thumb and a plurality of fingers, etc.). In some embodiments, the rotatable member 150 may include at least one recess 154 formed in an inner surface of the rotatable member 150. The at least one recess 154 may be configured to receive the at least one ramp element 134 therein in the unlocked configuration, as seen in FIG. 2A. In some embodiments, the rotatable member 150 may include at least one biasing surface 156 disposed on, extending along, and/or formed in the inner surface of the rotatable member 150. In at least some embodiments, the at least one biasing surface 156 may be circumferentially offset from and/or may be spaced apart from the at least one ramp element 134 in the unlocked configuration (e.g., FIG. 2A). In some embodiments, the at least one biasing surface 156 may be arcuate. In some embodiments, the at least one biasing surface 156 may be angled. Other configurations, including combinations thereof, are also contemplated. In some embodiments, the at least one biasing surface 156 may be configured to engage with and/or bias the at least one ramp element 134 and the plurality of beams 132 radially inward toward the central longitudinal axis of the polymeric tube 138 in the locked configuration, as seen in FIGS. 6A and 7. The rotatable member 150 is not shown in FIG. 7. As shown in FIGS. 6A and 7, in the locked configuration, the locking mechanism 140 and/or the rotatable member 150 may be configured to deflect the plurality of beams 132 in a radially inward direction. Accordingly, in the locked configuration, the plurality of beams 132 may squeeze the polymeric tube 138 against the elongate shaft 12 disposed within the lumen of the polymeric tube 138.

In some embodiments, the rotatable member 150 may include at least one second stop element 158 disposed therein and/or formed within an interior portion of the rotatable member 150. The at least one second stop element 158 may extend longitudinally along the inner surface of the rotatable member 150. In some embodiments, the at least one second stop element 158 may extend radially inward from the inner surface of the rotatable member 150. In some embodiments, the rotatable member 150 may include at least one third stop element 160 disposed therein and/or formed within the interior portion of the rotatable member 150. The at least one third stop element 160 may extend longitudinally along the inner surface of the rotatable member 150. In some embodiments, the at least one third stop element 160 may extend radially inward from the inner surface of the rotatable member 150. The at least one third stop element 160 may be circumferentially offset from the at least one second stop element 158.

In some embodiments, the at least one first stop element 136 of the locking portion 130 may be configured to engage the at least one second stop element 158 formed in the rotatable member 150 in the unlocked configuration, and the at least one first stop element 136 of the locking portion 130 may be configured to engage the at least one third stop element 160 formed in the rotatable member 150 in the locked configuration. In some embodiments, a first side of the at least one first stop element 136 of the locking portion 130 may be configured to engage the at least one second stop element 158 formed in the rotatable member 150 in the unlocked configuration, and a second side of the at least one first stop element 136 of the locking portion 130 may be configured to engage the at least one third stop element 160 formed in the rotatable member 150 in the locked configuration. The first side of the at least one first stop element 136 may be circumferentially spaced apart from the second side of the at least one first stop element 136.

In some embodiments, the at least one first stop element 136 may cooperate with the at least one second stop element 158 to form and/or define a “first hard stop” during rotation of the rotatable member 150 relative to and/or around the plurality of beams 132 when the locking portion 130 and/or the locking mechanism 140 is in the unlocked configuration (e.g., FIG. 2A). In some embodiments, the at least one first stop element 136 may cooperate with the at least one third stop element 160 to form and/or define a “second hard stop” during rotation of the rotatable member 150 relative to and/or around the plurality of beams 132 when the locking portion 130 and/or the locking mechanism 140 is in the locked configuration (e.g., FIG. 6A). Other configurations and/or other tactile feedback elements configured to communicate that the locking portion 130 and/or the locking mechanism 140 is in the unlocked configuration and/or the locked configuration are also contemplated.

In some embodiments, the rotatable member 150 may be configured to rotate between about 30 degrees and about 180 degrees to shift between the unlocked configuration (e.g., FIGS. 2-3) and/or the “first hard stop”, and the locked configuration (e.g., FIGS. 6-7) and/or the “second hard stop”. In some embodiments, the rotatable member 150 may be configured to rotate between about 40 degrees and about 120 degrees to shift between the unlocked configuration (e.g., FIGS. 2-3) and/or the “first hard stop”, and the locked configuration (e.g., FIGS. 6-7) and/or the “second hard stop”. In some embodiments, the rotatable member 150 may be configured to rotate between about 45 degrees and about 90 degrees to shift between the unlocked configuration (e.g., FIGS. 2-3) and/or the “first hard stop”, and the locked configuration (e.g., FIGS. 6-7) and/or the “second hard stop”. In some embodiments, the rotatable member 150 may be configured to rotate between about 50 degrees and about 75 degrees to shift between the unlocked configuration (e.g., FIGS. 2-3) and/or the “first hard stop”, and the locked configuration (e.g., FIGS. 6-7) and/or the “second hard stop”. In one non-limiting example, the rotatable member 150 may be configured to rotate about 60 degrees to shift between the unlocked configuration (e.g., FIGS. 2-3) and/or the “first hard stop”, and the locked configuration (e.g., FIGS. 6-7) and/or the “second hard stop”.

In some embodiments, the rotatable member 150 may include a locked indicator 162 disposed on the outer surface of the rotatable member 150. In some embodiments, the locked indicator 162 may be printed on the outer surface of the rotatable member 150. In some embodiments, the locked indicator 162 may be formed on the outer surface of the rotatable member 150. In one non-limiting example, the locked indicator 162 may be a raised feature extending radially outward from the outer surface of the rotatable member 150. In some embodiments, the locked indicator 162 may be formed in the outer surface of the rotatable member 150. In one non-limiting example, the locked indicator 162 may be a recessed feature extending radially inward from the outer surface of the rotatable member 150. Other configurations, including combinations thereof, are also contemplated.

In some embodiments, the rotatable member 150 may include an unlocked indicator 164 disposed on the outer surface of the rotatable member 150. In some embodiments, the unlocked indicator 164 may be printed on the outer surface of the rotatable member 150. In some embodiments, the unlocked indicator 164 may be formed on the outer surface of the rotatable member 150. In one non-limiting example, the unlocked indicator 164 may be a raised feature extending radially outward from the outer surface of the rotatable member 150. In some embodiments, the unlocked indicator 164 may be formed in the outer surface of the rotatable member 150. In one non-limiting example, the unlocked indicator 164 may be a recessed feature extending radially inward from the outer surface of the rotatable member 150. Other configurations, including combinations thereof, are also contemplated.

In some embodiments, the body portion 110 of the repositioning sheath 100 may include a pointer 118 (e.g., FIGS. 2, 6) configured to cooperate with the rotatable member 150 to visually communicate whether the locking portion 130 and/or the locking mechanism 140 is in the unlocked configuration (e.g., FIG. 2) or the locked configuration (e.g., FIG. 6). For example, if the pointer 118 is disposed adjacent to and/or points toward the unlocked indicator 164, as seen in FIG. 2, the locking portion 130 and/or the locking mechanism 140 may be clearly understood to be in the unlocked configuration, and if the pointer 118 is disposed adjacent to and/or points toward the locked indicator 162, as seen in FIG. 6, the locking portion 130 and/or the locking mechanism 140 may be clearly understood to be in the locked configuration. In some embodiments, the pointer 118 may be disposed on an outer surface of the body portion 110. In some embodiments, the pointer 118 may be printed on the outer surface of the body portion 110. In some embodiments, the pointer 118 may be formed on the outer surface of the body portion 110. In one non-limiting example, the pointer 118 may be a raised feature extending radially outward from the outer surface of the body portion 110. In some embodiments, the pointer 118 may be formed in the outer surface of the body portion 110. In one non-limiting example, the pointer 118 may be a recessed feature extending radially inward from the outer surface of the body portion 110. Other configurations, including combinations thereof, are also contemplated.

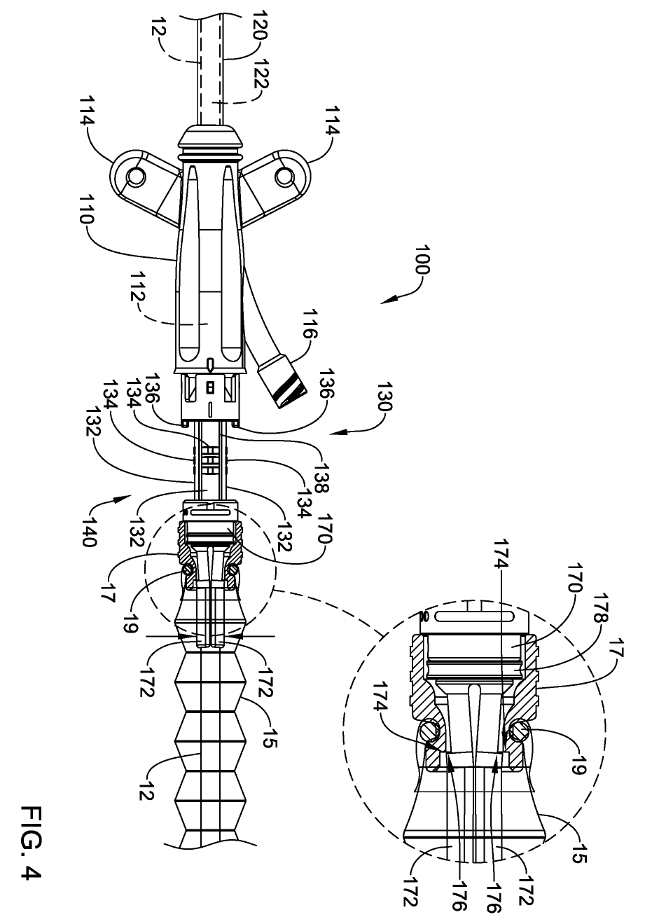

Turning back to FIGS. 3 and 4, a connection between the sterile sleeve 15 and the locking portion 130 may be seen in a partial cutaway and partial cross-sectional view. As discussed herein, the rotatable member 150 (e.g., FIG. 2) is not shown. The sterile sleeve 15 may be configured to releasably couple and/or releasably secure to the locking portion 130 of the repositioning sheath 100.

In some embodiments, the locking portion 130 of the repositioning sheath 100 may include a proximal attachment structure 170 configured to releasably couple and/or releasably secure the distal hub 17 of the sterile sleeve 15 to the locking portion 130 of the repositioning sheath 100. In some embodiments, the proximal attachment structure 170 may be fixedly attached to the locking portion 130 and/or the plurality of beams 132.

In some embodiments, the proximal attachment structure 170 may include a plurality of tabs 172 configured to shift between a radially expanded position (e.g., FIG. 3) and a radially collapsed position (e.g., FIG. 4). In some embodiments, the plurality of tabs 172 may comprise a pair of tabs (e.g., ref. 172) oppositely disposed from each other relative to the central longitudinal axis of the locking portion 130. Other configurations and/or quantities of tabs (e.g., ref. 172) are also contemplated. The proximal attachment structure 170 may include a lumen and/or an aperture extending longitudinally therethrough and/or through the plurality of tabs 172, such that the lumen and/or the aperture of the proximal attachment structure 170 is in communication with the lumen of the locking portion 130 and/or the lumen of the polymeric tube 138 (e.g., the elongate shaft 12 of the catheter 10 (e.g., the percutaneous circulatory support device) may extend through the lumen and/or the aperture of the proximal attachment structure 170 into the lumen of the locking portion 130 and/or the lumen of the polymeric tube 138).

The plurality of tabs 172 may be biased radially apart from each other and/or toward the radially expanded position (e.g., FIG. 3). In some embodiments, the plurality of tabs 172 may be self-biased radially apart from each other and/or toward the radially expanded position (e.g., FIG. 3). In some alternative embodiments, the plurality of tabs 172 may be spring biased radially apart from each other and/or toward the radially expanded position (e.g., FIG. 3). Other configurations are also contemplated.

In some embodiments, each tab of the plurality of tabs 172 may comprise a first ledge 174 configured to engage with the distal hub 17 of the sterile sleeve 15 to couple and/or secure the distal hub 17 of the sterile sleeve 15 to the locking portion 130 of the repositioning sheath 100 in the radially expanded position. In some embodiments, the distal hub 17 of the sterile sleeve 15 may comprise a second ledge 176 configured to engage with the first ledge 174 of each tab of the plurality of tabs 172 to couple and/or secure the distal hub 17 of the sterile sleeve 15 to the locking portion 130 of the repositioning sheath 100 in the radially expanded position. In at least some embodiments, the distal hub 17 of the sterile sleeve 15 may be configured to rotate relative to the locking portion 130 and/or the proximal attachment structure 170 of the repositioning sheath 100. Accordingly, relative rotational movement between the structures may be permitted to prevent adding torsion to or between the elongate shaft 12, the sterile sleeve 15, and/or the repositioning sheath 100.

In some embodiments, the proximal attachment structure 170 may include a sealing ring 178 extending circumferentially around the proximal attachment structure 170. In one example, the sealing ring 178 may have a generally X-shaped cross-section. In another example, the sealing ring 178 may be an O-ring. The sealing ring 178 may be configured to engage with an inner surface of the distal hub 17 to prevent fluid leakage and/or contamination. In some embodiments, the sterile sleeve 15 may be secured to the distal hub 17 with an O-ring 19. In one example, the O-ring 19 may be received within a circumferential slot formed in an outer surface of the distal hub 17. Other configurations are also contemplated.

In some embodiments, the plurality of tabs 172 may be deflectable radially inwards toward the radially collapsed position, such as by squeezing the plurality of tabs 172 toward each other and/or radially inward toward the central longitudinal axis of the locking portion 130, as seen in FIG. 4. In some embodiments, the sterile sleeve 15 may be sufficiently flexible to permit the user to apply radially inward force to the plurality of tabs 172 (e.g., to squeeze the plurality of tabs 172 toward each other and/or radially inward) within the sterile sleeve 15.

In some embodiments, the sterile sleeve 15 and/or the distal hub 17 of the sterile sleeve 15 may be configured to be snapped into engagement with and/or into securement with the proximal attachment structure 170 via relative translation of the distal hub 17 toward the proximal attachment structure 170. In some embodiments, the outer surface of the distal hub 17 may include a plurality of circumferential ribs, knurling, or other grip-enhancing elements configured to aid in urging the distal hub 17 into engagement with the proximal attachment structure 170.

In some embodiments, the plurality of tabs 172 may be configured to be squeezed and/or deflected radially toward each other to release the distal hub 17 of the sterile sleeve 15 from the locking portion 130 and/or the proximal attachment structure 170 of the repositioning sheath 100 in the radially collapsed position (e.g., FIG. 4). In at least some embodiments, coupling the distal hub and/or the sterile sleeve 15 to the locking portion 130 and/or the proximal attachment structure 170 of the repositioning sheath 100 may maintain the sterile field around the elongate shaft 12 of the catheter 10 (e.g., the percutaneous circulatory support device) while permitting selective axial and/or longitudinal adjustment of the elongate shaft 12 of the catheter 10 (e.g., the percutaneous circulatory support device) with respect to the repositioning sheath 100 in accordance with the disclosure.

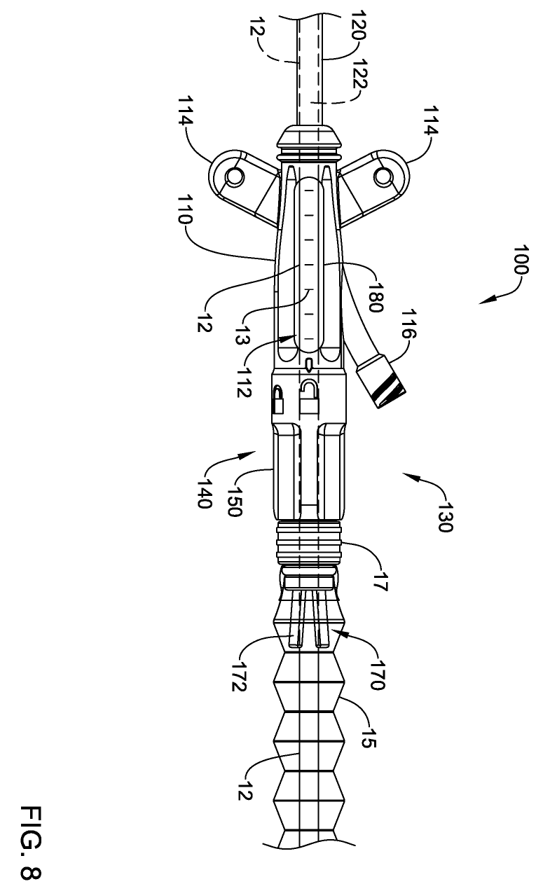

Turning now to FIG. 8, an alternative configuration of the repositioning sheath 100 is illustrated. Except as described below, the repositioning sheath 100 may be configured and/or constructed as described above, and description of such is not repeated in the interest of brevity. In some embodiments, the repositioning sheath 100 and/or the body portion 110 may include a viewing window 180 disposed within the body portion 110 distal of the locking portion 130 and proximal of the sheath 120. In some embodiments, the body portion 110 may comprise an aperture or an opening formed in a side wall of the body portion 110 to define the viewing window 180, and the viewing window 180 may comprise a transparent biocompatible material disposed within and/or filling the aperture or the opening formed in the side wall of the body portion 110. In some embodiments, the transparent biocompatible material may include a polymeric material, glass, or another suitable biocompatible material.

In some embodiments, the viewing window 180 may be formed on a top side of the body portion 110, as shown in FIG. 8. In some alternative embodiments, the viewing window 180 may be formed on a lateral side of the body portion 110. In some other alternative embodiments, the viewing window 180 may be formed on a bottom side of the body portion 110. In some embodiments, the viewing window 180 may comprise a plurality of viewing windows. In some embodiments, the plurality of viewing windows may be formed on one side of the body portion 110. In some embodiments, the plurality of viewing windows may be formed on multiple sides of the body portion 110. For example, the plurality of viewing windows may include a first viewing window formed on a first side of the body portion 110 and a second viewing window formed on a second side of the body portion 110 different from the first side of the body portion 110. Other configurations are also contemplated.

The viewing window 180 may be configured to permit visual observation and/or inspection of the elongate shaft 12 of the catheter 10 (e.g., the percutaneous circulatory support device) disposed within the lumen 112 of the body portion 110. In some embodiments, the elongate shaft 12 of the catheter 10 (e.g., the percutaneous circulatory support device) may include external markings 13 disposed thereon. In some embodiments, the external markings 13 may be disposed at equidistant intervals along the length of the elongate shaft 12. The external markings 13 may be visible through the viewing window 180 and may be used to identify a position of the elongate shaft 12 of the catheter 10 (e.g., the percutaneous circulatory support device) with respect to the repositioning sheath 100. In some embodiments, the body portion 110 and/or the viewing window 180 may include an index marking (not shown) used in combination with the external markings 13 to determine the position of the elongate shaft 12 of the catheter 10 (e.g., the percutaneous circulatory support device) with respect to the repositioning sheath 100. In some embodiments, the index marking may be a line, an arrow, some other indicia, or the like.

Additionally, the external markings 13 may be subsequently used, such as during a post-implantation check-up or inspection, to determine if the elongate shaft 12 of the catheter 10 (e.g., the percutaneous circulatory support device) has moved relative to the repositioning sheath 100 and/or to determine if the blood pump 50 has moved relative to the patient’s heart. Upon determining that the elongate shaft 12 and/or the blood pump 50 has moved from its prescribed, preferred, and/or optimal positioning, the external markings 13 may be used to reposition the elongate shaft 12 and/or the blood pump 50.

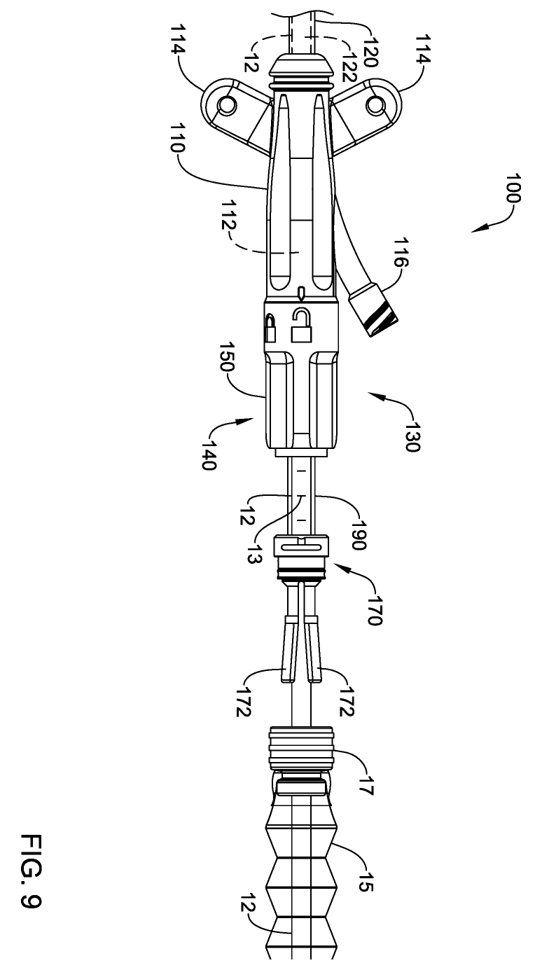

Another alternative embodiment of the repositioning sheath 100 is illustrated in FIG. 9. Except as described below, the repositioning sheath 100 may be configured and/or constructed as described above, and description of such is not repeated in the interest of brevity. In some embodiments, the repositioning sheath 100 and/or the body portion 110 may include a viewing tube 190 disposed proximal of and/or extending proximally from the locking portion 130. The viewing tube 190 may be fixedly attached to the locking portion 130. As shown, the proximal attachment structure 170 may be disposed at a proximal end of the viewing tube 190. The proximal attachment structure 170 may be fixedly attached to the proximal end of the viewing tube 190. The aperture or the opening formed in the proximal attachment structure 170 may be in communication with a lumen of the viewing tube 190.

In some embodiments, the viewing tube 190 may comprise a transparent biocompatible material. In some embodiments, the transparent biocompatible material may include a polymeric material or another suitable biocompatible material. The transparent biocompatible material forming the viewing tube 190 may be flexible to avoid adding “stiff” length to the repositioning sheath 100. Other configurations are also contemplated. Some suitable but non-limiting examples of materials for the viewing tube 190 may include polyamide, polyethylene (PE), polyethylene terephthalate (PET), polypropylene (PP), polytetrafluoroethylene (PTFE), fluorinated ethylene propylene (FEP), polyvinyl chloride (PVC), polyether block amides (PEBA), polycarbonate (PC), poly methyl methacrylate (PMMA), combinations thereof, etc.

The viewing tube 190 may be configured to permit visual observation and/or inspection of the elongate shaft 12 of the catheter 10 (e.g., the percutaneous circulatory support device) disposed within the lumen of the viewing tube 190. The external markings 13 disposed on the elongate shaft 12, as described above, may be visible through the viewing tube 190 and may be used to identify a position of the elongate shaft 12 of the catheter 10 (e.g., the percutaneous circulatory support device) with respect to the repositioning sheath 100. In some embodiments, the viewing tube 190 may include an index marking (not shown) used in combination with the external markings 13 to determine the position of the elongate shaft 12 of the catheter 10 (e.g., the percutaneous circulatory support device) with respect to the repositioning sheath 100. In some embodiments, the index marking may be a line, an arrow, some other indicia, or the like.

Additionally, the external markings 13 may be subsequently used, such as during a post-implantation check-up or inspection, to determine if the elongate shaft 12 of the catheter 10 (e.g., the percutaneous circulatory support device) has moved relative to the repositioning sheath 100 and/or to determine if the blood pump 50 has moved relative to the patient’s heart. Upon determining that the elongate shaft 12 and/or the blood pump 50 has moved from its prescribed, preferred, and/or optimal positioning, the external markings 13 may be used to reposition the elongate shaft 12 and/or the blood pump 50.

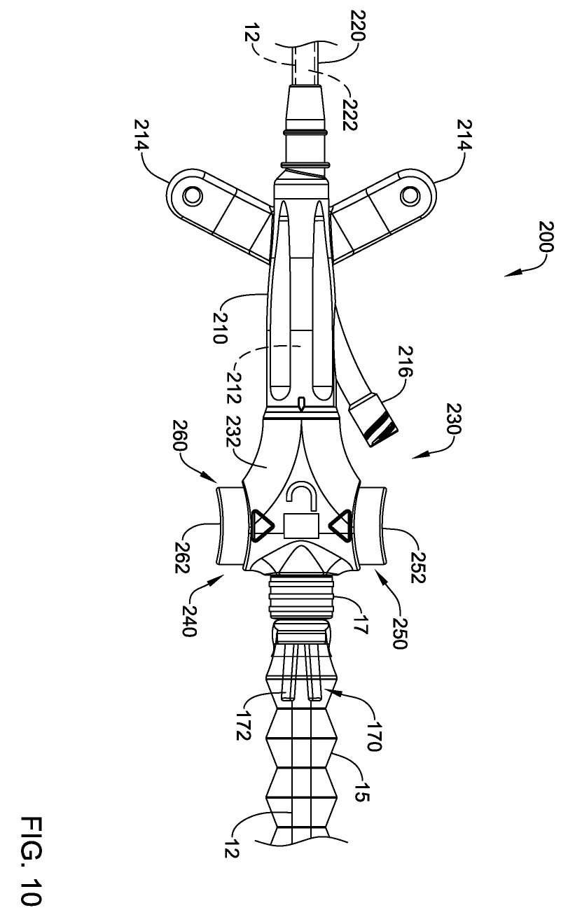

Turning now to FIGS. 10-17, the figures schematically illustrate selected aspects of a percutaneous circulatory support system including the catheter 10 (e.g., the percutaneous circulatory support device) positioned within a repositioning sheath 200 (e.g., a vascular access sheath, an introducer sheath, etc.) for use in the percutaneous circulatory support system. In some embodiments, the repositioning sheath 200 may be used for facilitating passage of various medical devices, such as the elongate shaft 12, the blood pump 50, etc., through a vascular access site of the subject (e.g., the patient) and into a blood vessel and/or the vasculature of the subject (e.g., the patient). In some embodiments, the repositioning sheath 200 may be configured to extend into the blood vessel and/or the vasculature of the subject (e.g., the patient) and allow the elongate shaft 12, the blood pump 50, and/or other suitable medical devices to pass therethrough. In at least some embodiments, during use, the elongate shaft 12 of the catheter 10 (e.g., the percutaneous circulatory support device) may be positionable within the repositioning sheath 200.

In some embodiments, the repositioning sheath 200 may comprise a body portion 210 including a lumen 212 extending therethrough and at least one wing 214 extending laterally from the body portion 210. The at least one wing 214 may be configured to be secured to the subject (e.g., the patient). In one non-limiting example, the at least one wing 214 may be sutured to skin of the subject (e.g., the patient). Other configurations are also contemplated. In some embodiments, the body portion 210 may include a hemostasis valve (not shown) disposed therein. The hemostasis valve may be configured to engage with the elongate shaft 12 of the catheter 10 (e.g., the percutaneous circulatory support device) to prevent and/or minimize blood leakage out of the repositioning sheath 200 during use. In at least some embodiments, the body portion 210 may include a side port 216 in fluid communication with the lumen 212 of the body portion 210. In some embodiments, the side port 216 may be fluidly coupled to a section of tubing (not shown), which may include a valve and/or a fluid port, in fluid communication with a source of fluid (not shown). In some embodiments, the side port 216 may be a guidewire port and/or may be configured to slidably receive a guidewire therein.

In some embodiments, the repositioning sheath 200 may comprise a sheath 220 extending distally from the body portion 210. The sheath 220 may include a lumen 222 in communication with the lumen 212 of the body portion 210. In at least some embodiments, the sheath 220 may be configured to fluidly engage with the blood vessel and/or the vasculature of the subject (e.g., the patient). In some embodiments, the sheath 220 may be fixedly attached to the body portion 210. In some embodiments, the sheath 220 may be integrally and/or monolithically formed with the body portion 210. In some alternative embodiments, the sheath 220 may be removably attached to the body portion 210.

In some embodiments, the sheath 220 may be configured to directly access and/or to extend directly into the blood vessel and/or the vasculature of the subject (e.g., the patient) via an arterial access site (e.g., a femoral access site, etc.). As such, in some embodiments, the sheath 220 may function as a vascular access sheath. In some embodiments, the blood pump 50 (e.g., FIG. 1) may be delivered to the heart of the patient via an arterial access site. In one non-limiting example, the blood pump 50 may be delivered to the heart of the patient via a femoral access site. However, utilizing a femoral access site may limit the mobility of the patient as movement of the elongate shaft 12 (extending out of a femoral access site) as the patient moves may result in inadvertent movement of the blood pump 50 in the heart of the patient. As such, to allow the patient to be ambulatory with the blood pump 50 operating in the heart of the patient, it may be advantageous to utilize an access site closer to the heart of the patient and/or not at a limb of the patient that moves relative to a core (e.g., relative to a chest) of the patient as the patient becomes ambulatory. In some examples, the axillary artery or subscapular artery may have a sufficient diameter to receive the blood pump and is close enough to the heart of the patient (e.g., located at the chest of the patient) to mitigate a likelihood of the elongate shaft 12 moving in a manner that would inadvertently change the position of the blood pump 50 in the heart of the patient as the patient becomes ambulatory.

In some alternative embodiments, the sheath 220 may be configured to fluidly couple with and/or be inserted into a graft (e.g., a port, an access tube, etc.) attached to, installed in, and/or fluidly coupled to the axillary artery or the subscapular artery. The graft may be attached to, installed in, and/or fluidly coupled to the axillary artery and/or the subscapular artery using a surgical technique or a percutaneous approach.

Using a surgical technique, the graft may be attached to the axillary artery or the subscapular artery and the blood pump 50 may be delivered to the heart of the patient via the axillary artery and/or the subscapular artery by inserting the blood pump 50 into the graft and advancing the blood pump 50 through the axillary artery and/or the subscapular artery to the heart. Use of the graft may facilitate providing an access site for inserting the blood pump 50 to the heart of the patient and through which the elongate shaft 12 may extend during operation of the blood pump 50, which may mitigate bleeding from the blood vessel and/or the vasculature and mitigate a likelihood of infection. Further, maintaining the graft at the access site may facilitate removal of the blood pump 50 from the patient after a period of time. Delivering the blood pump 50 to the heart of the patient via the axillary artery, the subscapular artery, and/or other suitable artery proximate the heart of the patient (e.g., at an upper extremity access site) may facilitate supporting a patient utilizing the blood pump 50 in an ambulatory state for an extended period of time (e.g., up to 2 weeks and/or beyond 2 weeks).

Utilizing a percutaneous approach for placing the graft may reduce bleeding and/or mitigate infection risks at the arterial access site for inserting the blood pump 50 into the vasculature of the patient. In some examples, a vascular access device with an elongate tubular frame and a graft or tubular extension extending from the elongate tubular frame may be inserted into an arterial system of the patient (e.g., via a radial artery or other suitable arterial access site) and delivered to a target location in the axillary artery or other suitable target location. Once the vascular access device is delivered to the target location, a cut or incision through the skin of the patient and the wall of the axillary artery of the patient may be made and the graft or tubular extension may be extended out through the wall of the axillary artery and out of the cut or incision through the skin of the patient. The blood pump 50 may then be delivered to the heart of the patient via the graft or tubular extension accessed in a subscapular region, for example, providing access to the axillary artery of the patient.

In some embodiments, a length of the sheath 220 may be related to and/or dependent upon how the sheath 220 is intended to engage with the vasculature of the patient. For example, when the sheath 220 is intended to be directly inserted into the vasculature of the patient and/or function as a vascular access sheath, the sheath 220 may be longer than if the sheath 220 is intended to be fluidly coupled with a graft in fluid communication with the axillary artery or the subscapular artery. In some embodiments, a form factor (e.g., shape, etc.) of the sheath 220 may change and/or be different to facilitate the different uses. In at least some embodiments, the sheath 220 may be formed from a polymeric material. Some suitable but non-limiting materials for the sheath 220, including polymeric materials, metallic materials, composites, and the like, are described below.

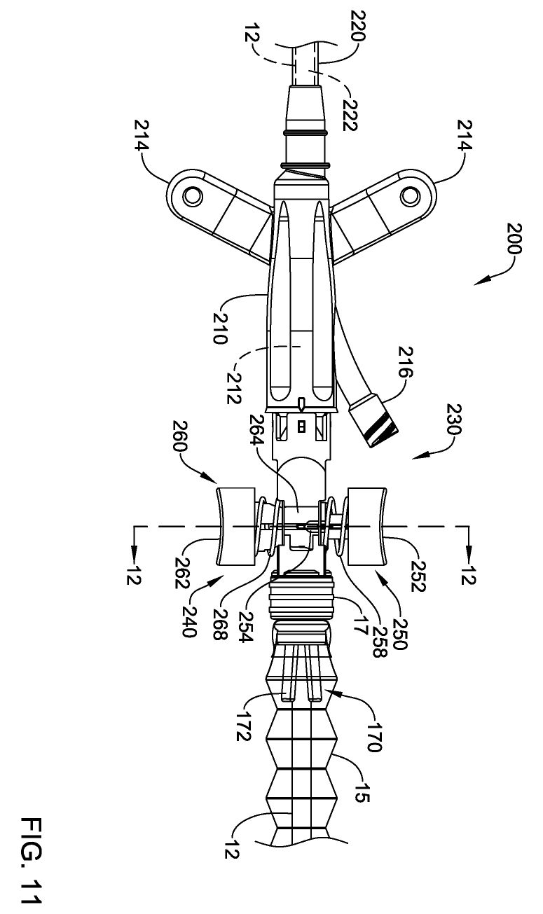

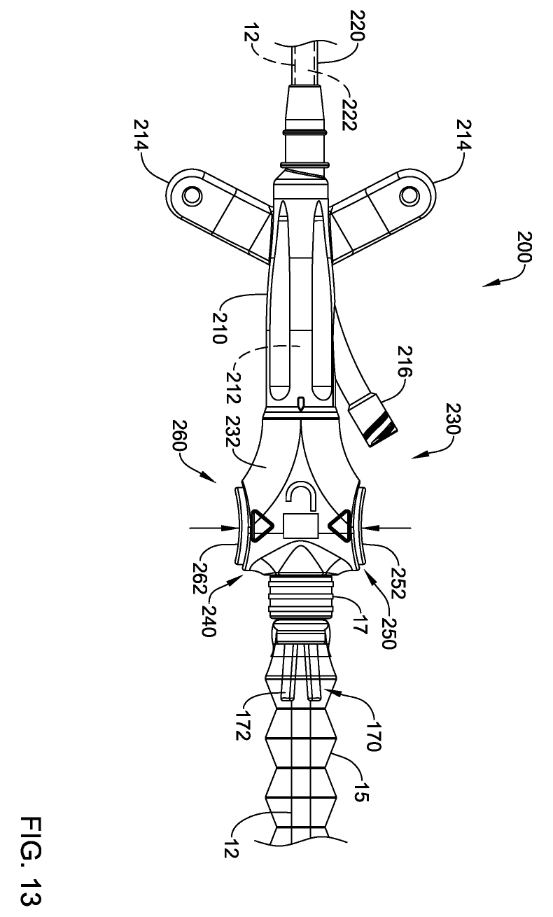

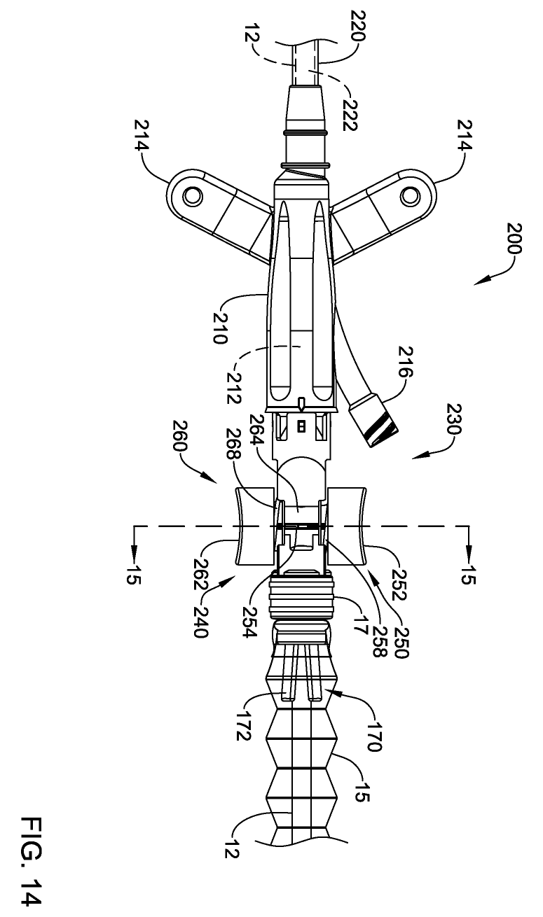

In some embodiments, the repositioning sheath 200 may comprise a locking portion 230 secured to the body portion 210. In some embodiments, the locking portion 230 may include a housing 232. In some embodiments, the locking portion 230 and/or the housing 232 may be fixedly attached to the body portion 210. In some embodiments, the locking portion 230 may be configured to shift between a locked configuration, as shown in FIGS. 10-12, and an unlocked configuration, as shown in FIGS. 13-15. To improve clarity and understanding, the housing 232 is not shown in FIGS. 11 and 14.

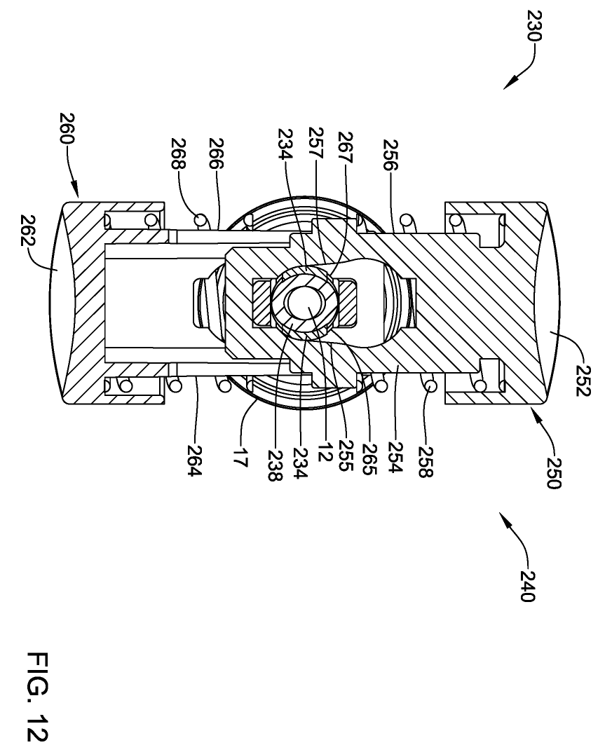

In some embodiments, the locking portion 230 and/or the housing 232 may include a lumen extending therethrough. In some embodiments, the lumen of the locking portion 230 and/or the housing 232 may be in communication with the lumen 212 of the body portion 210. In some embodiments, the locking portion 230 and/or the housing 232 may include a polymeric tube 238 (e.g., FIG. 12) disposed therein and/or disposed within the lumen of the locking portion 230 and/or the housing 232. The polymeric tube 238 may include a lumen in communication with the lumen 212 of the body portion 210. In some embodiments, the elongate shaft 12 of the catheter 10 (e.g., the percutaneous circulatory support device) may be slidably disposable within the lumen of the polymeric tube 238, the lumen 222 of the sheath 220, and the lumen 212 of the body portion 210.

In some embodiments, the locking portion 230 may include a locking mechanism 240 configured to selectively engage the polymeric tube 238 against the elongate shaft 12 of the catheter 10 (e.g., the percutaneous circulatory support device) disposed and/or positioned within the lumen of the polymeric tube 238 in the locked configuration (e.g., FIGS. 10-12). In some embodiments, the locking mechanism 240 may be configured to selectively squeeze the polymeric tube 238 against the elongate shaft 12 of the catheter 10 (e.g., the percutaneous circulatory support device) disposed and/or positioned within the lumen of the polymeric tube 238 in the locked configuration (e.g., FIGS. 10-12). In the unlocked configuration (e.g., FIGS. 13-15), the elongate shaft 12 of the catheter 10 (e.g., the percutaneous circulatory support device) may be slidably disposed within the lumen of the polymeric tube 238 and/or may be slidable through and/or within the locking portion 230 and/or the locking mechanism 240. Accordingly, in the unlocked configuration, the locking mechanism 240 does not squeeze the polymeric tube 238 against the elongate shaft 12 disposed within the lumen of the polymeric tube 238.

In the locked configuration, the locking portion 230 and/or the locking mechanism 240 may be configured to prevent relative axial translation of the elongate shaft 12 of the catheter 10 (e.g., the percutaneous circulatory support device) within and/or through the repositioning sheath 200, the locking portion 230, the lumen of the polymeric tube 238, and/or the locking mechanism 240. In some embodiments, the locking portion 230 and/or the locking mechanism 240 may be configured to permit the elongate shaft 12 of the catheter 10 (e.g., the percutaneous circulatory support device) to slide therethrough in the unlocked configuration, and the locking mechanism 240 may be configured to prevent the elongate shaft 12 of the catheter 10 (e.g., the percutaneous circulatory support device) from sliding therethrough in the locked configuration.

In some embodiments, the polymeric tube 238 may be formed from a polymeric material such that when engaged with the elongate shaft 12 of the catheter 10 (e.g., the percutaneous circulatory support device), a frictional force between the polymeric tube 238 and the elongate shaft 12 of the catheter 10 (e.g., the percutaneous circulatory support device) is sufficient to prevent relative axial translation of the elongate shaft 12 of the catheter 10 (e.g., the percutaneous circulatory support device) within and/or through the repositioning sheath 200, the locking portion 230, the lumen of the polymeric tube 238, and/or the locking mechanism 240 during normal patient movement. In some examples, the frictional force may be at least 50 grams-force (490.33 milliNewton (mN)). In some examples, the frictional force may be at least 75 grams-force (735.50 mN). In one preferred but non-limiting example, the frictional force may be at least 100 grams-force (980.67 mN). In some examples, the frictional force may be at least 125 grams-force (1225.83 mN). In some examples, the frictional force may be at least 150 grams-force (1471.00 mN). In some examples, the frictional force may be at least 175 grams-force (1716.16 mN). In some examples, the frictional force may be at least 200 grams-force (1961.33 mN). In some examples, the frictional force may be between about 25 grams-force (245.17 mN) and about 250 grams-force (2451.66 mN). In some examples, the frictional force may be between about 50 grams-force (490.33 mN) and about 200 grams-force (1961.33 mN). In some examples, the frictional force may be between about 75 grams-force (735.50 mN) and about 150 grams-force (1471.00 mN). In some examples, the frictional force may be between about 100 grams-force (980.67 mN) and about 125 grams-force (1225.83 mN). Other examples and/or values are also contemplated. In one non-limiting example, the polymeric tube 238 may comprise and/or may be formed from silicone. Some other suitable but non-limiting polymeric materials for the polymeric tube 238 are described below.

In some embodiments, the locking mechanism 240 may include a first button structure 250 and a second button structure 260 disposed opposite the first button structure 250 relative to a central longitudinal axis of the locking portion 230 and/or the polymeric tube 238. The first button structure 250 and/or the second button structure 260 may be at least partially disposed within the housing 232 of the locking portion 230.

In some embodiments, the first button structure 250 may include a user-engageable portion 252 (e.g., a first force receiving surface) disposed on an outward facing surface of the first button structure 250. In at least some embodiments, the user-engageable portion 252 (e.g., the first force receiving surface) of the first button structure 250 may be configured to be engaged by a user (e.g., by the user’s finger or thumb). In some embodiments, the user-engageable portion 252 (e.g., the first force receiving surface) of the first button structure 250 may be configured to be selectively translated radially inward toward the central longitudinal axis of the locking portion 230 and/or the polymeric tube 238 by the user.

In some embodiments, the second button structure 260 may include a user-engageable portion 262 (e.g., a second force receiving surface) disposed on an outward facing surface of the second button structure 260. In at least some embodiments, the user-engageable portion 262 (e.g., a second force receiving surface) of the second button structure 260 may be configured to be engaged by a user (e.g., by the user’s finger or thumb). In some embodiments, the user-engageable portion 262 (e.g., a second force receiving surface) of the second button structure 260 may be configured to be selectively translated radially inward toward the central longitudinal axis of the locking portion 230 and/or the polymeric tube 238 by the user.

In some embodiments, the user-engageable portion 252 (e.g., the first force receiving surface) of the first button structure 250 and the user-engageable portion 262 (e.g., a second force receiving surface) of the second button structure 260 may be translatable toward each other by the user to shift the locking portion 230 and/or the locking mechanism 240 from the locked configuration (e.g., FIGS. 10-12) toward and/or to the unlocked configuration (e.g., FIGS. 13-15).

In some embodiments, the second button structure 260 may be slidably engaged with the first button structure 250. In some embodiments, the first button structure 250 may comprise an upper slide 254 (e.g., FIGS. 11-12) and a lower slide 256 (e.g., FIG. 12) extending away from the user-engageable portion 252 (e.g., the first force receiving surface) of the first button structure 250 on opposite sides of the central longitudinal axis of the locking portion 230 and/or the polymeric tube 238. In some embodiments, the second button structure 260 may comprise an upper slide 264 (e.g., FIGS. 11-12) and a lower slide 266 (e.g., FIG. 12) extending away from the user-engageable portion 262 (e.g., a second force receiving surface) of the second button structure 260 on opposite sides of the central longitudinal axis of the locking portion 230 and/or the polymeric tube 238. In at least some embodiments, the upper slide 254 of the first button structure 250 may be slidably engaged with the upper slide 264 of the second button structure 260, and the lower slide 256 of the first button structure 250 may be slidably engaged with the lower slide 266 of the second button structure 260. Other configurations are also contemplated.

In some embodiments, the second button structure 260 may be oriented substantially parallel to the first button structure 250. In some embodiments, the upper slide 254 of the first button structure 250 may be oriented parallel to the upper slide 264 of the second button structure 260, and the lower slide 256 of the first button structure 250 may be oriented parallel to the lower slide 266 of the second button structure 260. In some embodiments, the first button structure 250 may be oriented laterally with respect to and/or may be oriented substantially perpendicular to the central longitudinal axis of the locking portion 230 and/or the polymeric tube 238. In some embodiments, the second button structure 260 may be oriented laterally with respect to and/or may be oriented substantially perpendicular to the central longitudinal axis of the locking portion 230 and/or the polymeric tube 238. In some embodiments, the upper slide 254 of the first button structure 250 and the upper slide 264 of the second button structure 260 may be oriented laterally with respect to and/or may be oriented substantially perpendicular to the central longitudinal axis of the locking portion 230 and/or the polymeric tube 238, and the lower slide 256 of the first button structure 250 and the lower slide 266 of the second button structure 260 may be oriented laterally with respect to and/or may be oriented substantially perpendicular to the central longitudinal axis of the locking portion 230 and/or the polymeric tube 238. Other configurations are also contemplated.

In some embodiments, the upper slide 254 of the first button structure 250 may include an angled surface 255 configured to bias and/or squeeze the polymeric tube 238 toward and/or against the elongate shaft 12 of the catheter 10 (e.g., the percutaneous circulatory support device) disposed therein in the locked configuration, as seen in FIG. 12. In some embodiments, the lower slide 256 of the first button structure 250 may include an angled surface 257 configured to bias and/or squeeze the polymeric tube 238 toward and/or against the elongate shaft 12 of the catheter 10 (e.g., the percutaneous circulatory support device) disposed therein in the locked configuration, as seen in FIG. 12. In some embodiments, the upper slide 264 of the second button structure 260 may include an angled surface 265 configured to bias and/or squeeze the polymeric tube 238 toward and/or against the elongate shaft 12 of the catheter 10 (e.g., the percutaneous circulatory support device) disposed therein in the locked configuration, as seen in FIG. 12. In some embodiments, the lower slide 266 of the second button structure 260 may include an angled surface 267 configured to bias and/or squeeze the polymeric tube 238 toward and/or against the elongate shaft 12 of the catheter 10 (e.g., the percutaneous circulatory support device) disposed therein in the locked configuration, as seen in FIG. 12.

In some embodiments, the angled surface 255 of the upper slide 254 and the angled surface 257 of the lower slide 256 of the first button structure 250 may be configured to bias and/or squeeze a plurality of longitudinal beams 234 (e.g., FIG. 12) of the locking portion 230 disposed between the angled surface 255 of the upper slide 254 and the angled surface 257 of the lower slide 256 of the first button structure 250 and the polymeric tube 238 toward and/or against the polymeric tube 238 to engage and/or squeeze the elongate shaft 12 of the catheter 10 (e.g., the percutaneous circulatory support device) disposed therein in the locked configuration. In some embodiments, the angled surface 265 of the upper slide 264 and the angled surface 267 of the lower slide 266 of the second button structure 260 may be configured to bias and/or squeeze the plurality of longitudinal beams 234 (e.g., FIG. 12) of the locking portion 230 disposed between the angled surface 265 of the upper slide 264 and the angled surface 267 of the lower slide 266 of the second button structure 260 and the polymeric tube 238 toward and/or against the polymeric tube 238 to engage and/or squeeze the elongate shaft 12 of the catheter 10 (e.g., the percutaneous circulatory support device) disposed therein in the locked configuration.

In some alternative embodiments, the angled surface 255 of the upper slide 254 and the angled surface 257 of the lower slide 256 of the first button structure 250 may be configured to be in direct contact with and/or may be configured to directly bias and/or squeeze the polymeric tube 238 toward and/or against the elongate shaft 12 of the catheter 10 (e.g., the percutaneous circulatory support device) disposed therein in the locked configuration. In some alternative embodiments, the angled surface 265 of the upper slide 264 and the angled surface 267 of the lower slide 266 of the second button structure 260 may be configured to be in direct contact with and/or may be configured to directly bias and/or squeeze the polymeric tube 238 toward and/or against the elongate shaft 12 of the catheter 10 (e.g., the percutaneous circulatory support device) disposed therein in the locked configuration.

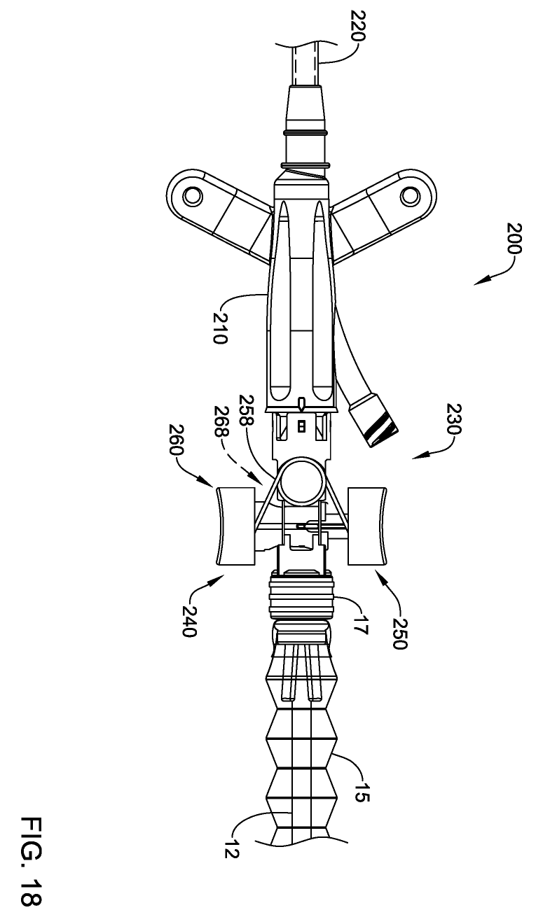

In some embodiments, the locking mechanism 240 may include a first spring 258 configured to bias the user-engageable portion 252 (e.g., the first force receiving surface) of the first button structure 250 away from the central longitudinal axis of the locking portion 230 and/or the polymeric tube 238. In some embodiments, the locking mechanism 240 may include a second spring 268 configured to bias the user-engageable portion 262 (e.g., the second force receiving surface) of the second button structure 260 away from the central longitudinal axis of the locking portion 230 and/or the polymeric tube 238. In some embodiments, the locking portion 230 and/or the locking mechanism 240 may be self-biased toward the locked configuration (e.g., FIGS. 10-12). In some embodiments, the first spring 258 may be disposed between the plurality of longitudinal beams 234 of the locking portion 230 and the first button structure 250. In some embodiments, the second spring 268 may be disposed between the plurality of longitudinal beams 234 of the locking portion 230 and the second button structure 260. Other configurations are also contemplated. In at least some embodiments, the first spring 258 and/or the second spring 268 may be a coil spring. In some embodiments, the first spring 258 may be held in compression between the plurality of longitudinal beams 234 of the locking portion 230 and the first button structure 250. In some embodiments, the second spring 268 may be held in compression between the plurality of longitudinal beams 234 of the locking portion 230 and the second button structure 260. Other configurations are also contemplated. For example, in some alternative embodiments, the first spring 258 and/or the second spring 268 may be a torsion spring, as seen in FIG. 18. In some embodiments, in the absence of a radially inward force applied to the user-engageable portion 252 (e.g., the first force receiving surface) of the first button structure 250 and the user-engageable portion 262 (e.g., the second force receiving surface) of the second button structure 260, the locking portion 230 and/or the locking mechanism 240 may be configured to automatically shift toward and/or to the locked configuration.

Returning now to FIGS. 13-15, the repositioning sheath 200 is illustrated with the locking portion 230 and/or the locking mechanism 240 in the unlocked configuration. In the unlocked configuration illustrated, the first button structure 250 and/or the user-engageable portion 252 (e.g., the first force receiving surface) of the first button structure 250 has been selectively translated radially inward toward the central longitudinal axis of the locking portion 230 and/or the polymeric tube 238 by the user. Similarly, the second button structure 260 and/or the user-engageable portion 262 (e.g., the second force receiving surface) of the second button structure 260 has been selectively translated radially inward toward the central longitudinal axis of the locking portion 230 and/or the polymeric tube 238 by the user. The first button structure 250 and the second button structure 260 may cooperate to shift the locking portion 230 and/or the locking mechanism 240 from the locked configuration (e.g., FIGS. 10-12) toward and/or to the unlocked configuration (e.g., FIGS. 13-15).

In some embodiments, the first button structure 250 and the second button structure 260 may be configured to translate and/or slide radially inward toward each other to shift the locking portion 230 and/or the locking mechanism 240 from the locked configuration (e.g., FIGS. 10-12) toward and/or to the unlocked configuration (e.g., FIGS. 13-15).

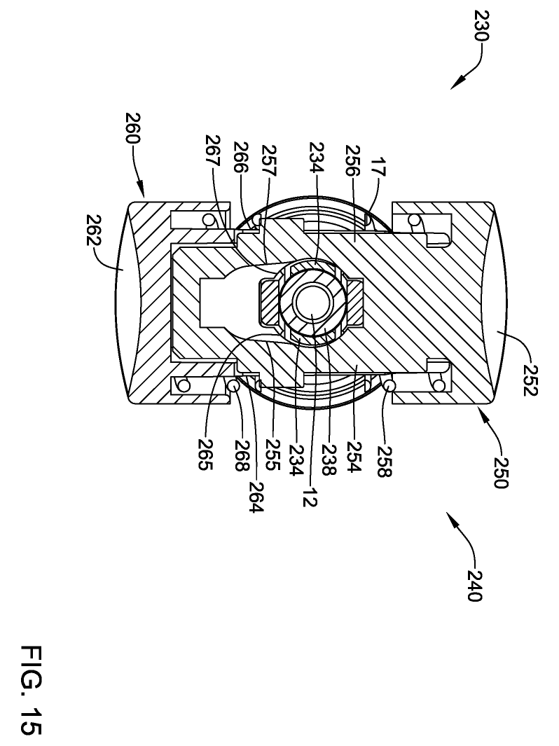

In some embodiments, the angled surface 255 of the upper slide 254 and the angled surface 257 of the lower slide 256 of the first button structure 250 may be configured to avoid biasing and/or squeezing the polymeric tube 238 toward and/or against the elongate shaft 12 of the catheter 10 (e.g., the percutaneous circulatory support device) disposed therein in the unlocked configuration, as seen in FIG. 15. In some embodiments, the angled surface 255 of the upper slide 254 and the angled surface 257 of the lower slide 256 of the first button structure 250 may be configured to avoid biasing and/or squeezing the plurality of longitudinal beams 234 toward and/or against the polymeric tube 238 to engage and/or squeeze the elongate shaft 12 of the catheter 10 (e.g., the percutaneous circulatory support device) disposed therein in the unlocked configuration. In some embodiments, in the unlocked configuration, the polymeric tube 238 may be radially spaced apart from the elongate shaft 12 of the catheter 10 (e.g., the percutaneous circulatory support device) disposed therein. In some embodiments, the angled surface 255 of the upper slide 254 and the angled surface 257 of the lower slide 256 of the first button structure 250 may be radially spaced apart from the polymeric tube 238 and/or the plurality of longitudinal beams 234 in the unlocked configuration.

In some embodiments, the angled surface 265 of the upper slide 264 and the angled surface 267 of the lower slide 266 of the second button structure 260 may be configured to avoid biasing and/or squeezing the polymeric tube 238 toward and/or against the elongate shaft 12 of the catheter 10 (e.g., the percutaneous circulatory support device) disposed therein in the unlocked configuration, as seen in FIG. 15. In some embodiments, the angled surface 265 of the upper slide 264 and the angled surface 267 of the lower slide 266 of the second button structure 260 may be configured to avoid biasing and/or squeezing the plurality of longitudinal beams 234 toward and/or against the polymeric tube 238 to engage and/or squeeze the elongate shaft 12 of the catheter 10 (e.g., the percutaneous circulatory support device) disposed therein in the unlocked configuration. In some embodiments, in the unlocked configuration, the polymeric tube 238 may be radially spaced apart from the elongate shaft 12 of the catheter 10 (e.g., the percutaneous circulatory support device) disposed therein. In some embodiments, the angled surface 265 of the upper slide 264 and the angled surface 267 of the lower slide 266 of the second button structure 260 may be radially spaced apart from the polymeric tube 238 and/or the plurality of longitudinal beams 234 in the unlocked configuration.

In some embodiments, the sterile sleeve 15 and the locking portion 230 may be connectable together and/or to each other in the same way as the sterile sleeve 15 and the repositioning sheath 100 above, for example, as shown in FIGS. 3-4. The sterile sleeve 15 may be configured to releasably couple and/or releasably secure to the locking portion 230 of the repositioning sheath 200, as shown in FIGS. 10-18.

In some embodiments, the locking portion 230 of the repositioning sheath 200 may include the proximal attachment structure 170, as described above, configured to releasably couple and/or releasably secure the distal hub 17 of the sterile sleeve 15 to the locking portion 230 of the repositioning sheath 200. In some embodiments, the proximal attachment structure 170 may be fixedly attached to the locking portion 230. The proximal attachment structure 170 may have the same form and/or construction as that described above with respect to FIGS. 3-4. As such, the same reference numbers are used for the proximal attachment structure 170 of the repositioning sheath 200.

In some embodiments, the proximal attachment structure 170 may include a plurality of tabs 172 configured to shift between a radially expanded position (e.g., FIG. 3) and a radially collapsed position (e.g., FIG. 4). In some embodiments, the plurality of tabs 172 may comprise a pair of tabs (e.g., ref. 172) oppositely disposed from each other relative to the central longitudinal axis of the locking portion 230. Other configurations and/or quantities of tabs (e.g., ref. 172) are also contemplated. The proximal attachment structure 170 may include a lumen and/or an aperture extending longitudinally therethrough and/or through the plurality of tabs 172, such that the lumen and/or the aperture of the proximal attachment structure 170 is in communication with the lumen of the locking portion 230 and/or the lumen of the polymeric tube 238 (e.g., the elongate shaft 12 of the catheter 10 (e.g., the percutaneous circulatory support device) may extend through the lumen and/or the aperture of the proximal attachment structure 170 into the lumen of the locking portion 230 and/or the lumen of the polymeric tube 238).

The plurality of tabs 172 may be biased radially apart from each other and/or toward the radially expanded position (e.g., FIG. 3). In some embodiments, the plurality of tabs 172 may be self-biased radially apart from each other and/or toward the radially expanded position (e.g., FIG. 3). In some alternative embodiments, the plurality of tabs 172 may be spring biased radially apart from each other and/or toward the radially expanded position (e.g., FIG. 3). Other configurations are also contemplated.

In some embodiments, each tab of the plurality of tabs 172 may comprise a first ledge 174 configured to engage with the distal hub 17 of the sterile sleeve 15 to couple and/or secure the distal hub 17 of the sterile sleeve 15 to the locking portion 230 of the repositioning sheath 200 in the radially expanded position. In some embodiments, the distal hub 17 of the sterile sleeve 15 may comprise a second ledge 176 configured to engage with the first ledge 174 of each tab of the plurality of tabs 172 to couple and/or secure the distal hub 17 of the sterile sleeve 15 to the locking portion 230 of the repositioning sheath 200 in the radially expanded position. In at least some embodiments, the distal hub 17 of the sterile sleeve 15 may be configured to rotate relative to the locking portion 230 and/or the proximal attachment structure 170 of the repositioning sheath 200. Accordingly, relative rotational movement between the structures may be permitted to prevent adding torsion to or between the elongate shaft 12, the sterile sleeve 15, and/or the repositioning sheath 200.

In some embodiments, the proximal attachment structure 170 may include a sealing ring 178 (e.g., FIGS. 3-4) extending circumferentially around the proximal attachment structure 170. In one example, the sealing ring 178 may have a generally X-shaped cross-section. In another example, the sealing ring 178 may be an O-ring. The sealing ring 178 may be configured to engage with an inner surface of the distal hub 17 to prevent fluid leakage and/or contamination. In some embodiments, the sterile sleeve 15 may be secured to the distal hub 17 with an O-ring 19. In one example, the O-ring 19 may be received within a circumferential slot formed in an outer surface of the distal hub 17. Other configurations are also contemplated.

In some embodiments, the plurality of tabs 172 may be deflectable radially inwards toward the radially collapsed position, such as by squeezing the plurality of tabs 172 toward each other and/or radially inward toward the central longitudinal axis of the locking portion 130, as seen in FIG. 4. In some embodiments, the sterile sleeve 15 may be sufficiently flexible to permit the user to apply radially inward force to the plurality of tabs 172 (e.g., to squeeze the plurality of tabs 172 toward each other and/or radially inward) within the sterile sleeve 15.

In some embodiments, the sterile sleeve 15 and/or the distal hub 17 of the sterile sleeve 15 may be configured to be snapped into engagement with and/or into securement with the proximal attachment structure 170 via relative translation of the distal hub 17 toward the proximal attachment structure 170. In some embodiments, the outer surface of the distal hub 17 may include a plurality of circumferential ribs, knurling, or other grip-enhancing elements configured to aid in urging the distal hub 17 into engagement with the proximal attachment structure 170.

In some embodiments, the plurality of tabs 172 may be configured to be squeezed and/or deflected radially toward each other to release the distal hub 17 of the sterile sleeve 15 from the locking portion 130 and/or the proximal attachment structure 170 of the repositioning sheath 100 in the radially collapsed position (e.g., FIG. 4). In at least some embodiments, coupling the distal hub and/or the sterile sleeve 15 to the locking portion 230 and/or the proximal attachment structure 170 of the repositioning sheath 200 may maintain the sterile field around the elongate shaft 12 of the catheter 10 (e.g., the percutaneous circulatory support device) while permitting selective axial and/or longitudinal adjustment of the elongate shaft 12 of the catheter 10 (e.g., the percutaneous circulatory support device) with respect to the repositioning sheath 200 in accordance with the disclosure.

In some alternative embodiments, the repositioning sheath 200 may comprise a viewing window (e.g., ref. 180; FIG. 8) disposed within the body portion 210 distal of the locking portion 230 and proximal of the sheath 220. In some embodiments, the body portion 210 may comprise an aperture or an opening formed in a side wall of the body portion 210 to define the viewing window (e.g., ref. 180; FIG. 8), and the viewing window (e.g., ref. 180; FIG. 8) may comprise a transparent biocompatible material disposed within and/or filling the aperture or the opening formed in the side wall of the body portion 210. In some embodiments, the transparent biocompatible material may include a polymeric material, glass, or another suitable biocompatible material.

In some embodiments, the viewing window (e.g., ref. 180; FIG. 8) may be formed on a top side of the body portion 210, similar to the configuration shown in FIG. 8. In some alternative embodiments, the viewing window (e.g., ref. 180; FIG. 8) may be formed on a lateral side of the body portion 210. In some other alternative embodiments, the viewing window (e.g., ref. 180; FIG. 8) may be formed on a bottom side of the body portion 210. In some embodiments, the viewing window (e.g., ref. 180; FIG. 8) may comprise a plurality of viewing windows. In some embodiments, the plurality of viewing windows may be formed on one side of the body portion 210. In some embodiments, the plurality of viewing windows may be formed on multiple sides of the body portion 210. For example, the plurality of viewing windows may include a first viewing window formed on a first side of the body portion 210 and a second viewing window formed on a second side of the body portion 210 different from the first side of the body portion 110. Other configurations are also contemplated.

The viewing window (e.g., ref. 180; FIG. 8) may be configured to permit visual observation and/or inspection of the elongate shaft 12 of the catheter 10 (e.g., the percutaneous circulatory support device) disposed within the lumen 212 of the body portion 210. In some embodiments, the elongate shaft 12 of the catheter 10 (e.g., the percutaneous circulatory support device) may include external markings (e.g., ref. 13) disposed thereon. In some embodiments, the external markings (e.g., ref. 13) may be disposed at equidistant intervals along the length of the elongate shaft 12. The external markings (e.g., ref. 13) may be visible through the viewing window (e.g., ref. 180; FIG. 8) and may be used to identify a position of the elongate shaft 12 of the catheter 10 (e.g., the percutaneous circulatory support device) with respect to the repositioning sheath 200. In some embodiments, the body portion 210 and/or the viewing window (e.g., ref. 180; FIG. 8) may include an index marking (not shown) used in combination with the external markings (e.g., ref. 13) to determine the position of the elongate shaft 12 of the catheter 10 (e.g., the percutaneous circulatory support device) with respect to the repositioning sheath 200. In some embodiments, the index marking may be a line, an arrow, some other indicia, or the like.

Additionally, the external markings (e.g., ref. 13) may be subsequently used, such as during a post-implantation check-up or inspection, to determine if the elongate shaft 12 of the catheter 10 (e.g., the percutaneous circulatory support device) has moved relative to the repositioning sheath 200 and/or to determine if the blood pump 50 has moved relative to the patient’s heart. Upon determining that the elongate shaft 12 and/or the blood pump 50 has moved from its prescribed, preferred, and/or optimal positioning, the external markings (e.g., ref. 13) may be used to reposition the elongate shaft 12 and/or the blood pump 50.

In another alternative embodiment of the repositioning sheath 200 and/or the body portion 210 may include a viewing tube (e.g., ref. 190; FIG. 9) disposed proximal of and/or extending proximally from the locking portion 230. The viewing tube (e.g., ref. 190; FIG. 9) may be fixedly attached to the locking portion 230. As shown, the proximal attachment structure 170 may be disposed at a proximal end of the viewing tube (e.g., ref. 190; FIG. 9). The proximal attachment structure 170 may be fixedly attached to the proximal end of the viewing tube (e.g., ref. 190; FIG. 9). The aperture or the opening formed in the proximal attachment structure 170 may be in communication with a lumen of the viewing tube (e.g., ref. 190; FIG. 9).

In some embodiments, the viewing tube (e.g., ref. 190; FIG. 9) may comprise a transparent biocompatible material. In some embodiments, the transparent biocompatible material may include a polymeric material or another suitable biocompatible material. The transparent biocompatible material forming the viewing tube (e.g., ref. 190; FIG. 9) may be flexible to avoid adding “stiff” length to the repositioning sheath 200. Other configurations are also contemplated. Some suitable but non-limiting examples of materials for the viewing tube (e.g., ref. 190; FIG. 9) may include polyamide, polyethylene (PE), polyethylene terephthalate (PET), polypropylene (PP), polytetrafluoroethylene (PTFE), fluorinated ethylene propylene (FEP), polyvinyl chloride (PVC), polyether block amides (PEBA), polycarbonate (PC), poly methyl methacrylate (PMMA), combinations thereof, etc.

The viewing tube (e.g., ref. 190; FIG. 9) may be configured to permit visual observation and/or inspection of the elongate shaft 12 of the catheter 10 (e.g., the percutaneous circulatory support device) disposed within the lumen of the viewing tube (e.g., ref. 190; FIG. 9). The external markings (e.g., ref. 13) disposed on the elongate shaft 12, as described above, may be visible through the viewing tube (e.g., ref. 190; FIG. 9) and may be used to identify a position of the elongate shaft 12 of the catheter 10 (e.g., the percutaneous circulatory support device) with respect to the repositioning sheath 200. In some embodiments, the viewing tube (e.g., ref. 190; FIG. 9) may include an index marking (not shown) used in combination with the external markings (e.g., ref. 13) to determine the position of the elongate shaft 12 of the catheter 10 (e.g., the percutaneous circulatory support device) with respect to the repositioning sheath 200. In some embodiments, the index marking may be a line, an arrow, some other indicia, or the like.

Additionally, the external markings (e.g., ref. 13) may be subsequently used, such as during a post-implantation check-up or inspection, to determine if the elongate shaft 12 of the catheter 10 (e.g., the percutaneous circulatory support device) has moved relative to the repositioning sheath 200 and/or to determine if the blood pump 50 has moved relative to the patient’s heart. Upon determining that the elongate shaft 12 and/or the blood pump 50 has moved from its prescribed, preferred, and/or optimal positioning, the external markings (e.g., ref. 13) may be used to reposition the elongate shaft 12 and/or the blood pump 50.

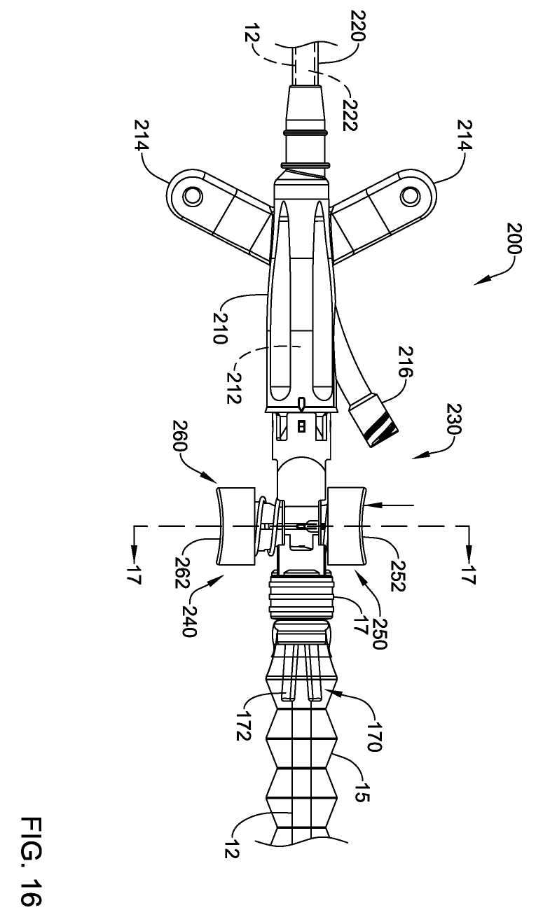

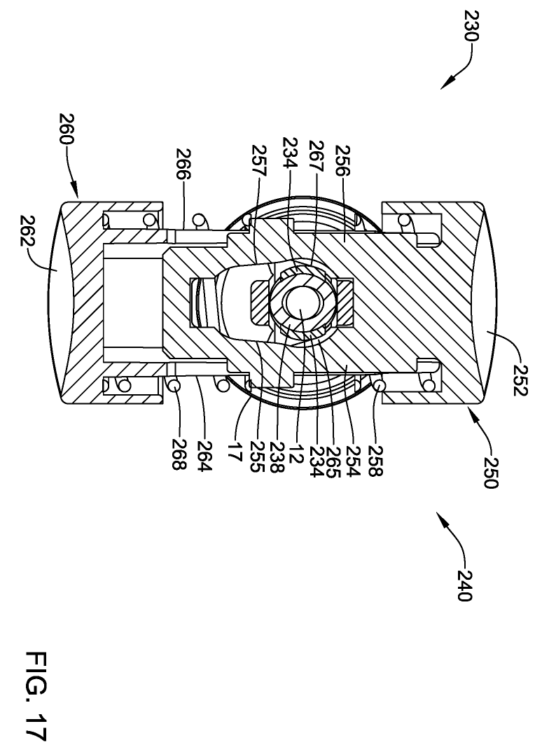

Turning now to FIGS. 16-17, the repositioning sheath 200 is shown in a configuration with only the first button structure 250 translated toward the central longitudinal axis of the locking portion 230 and/or the polymeric tube 238 by the user (e.g., the second button structure 260 has not been translated toward the central longitudinal axis of the locking portion 230 and/or the polymeric tube 238 by the user). As seen in FIG. 17, the first button structure 250 has been shifted toward and/or to the unlocked configuration, but the second button structure 260 remains in the locked configuration. As such, the locking portion 230 and/or the locking mechanism 240 remains in the locked configuration. That is, the elongate shaft 12 of the catheter 10 (e.g., the percutaneous circulatory support device) disposed within the repositioning sheath 200 and/or the polymeric tube 238 is still axially secured relative to the repositioning sheath 200 (e.g., the elongate shaft 12 cannot translate axially within and/or through the repositioning sheath 200). Accordingly, the first button structure 250 and the second button structure 260 may be configured to cooperate to shift the locking portion 230 and/or the locking mechanism 240 from the locked configuration (e.g., FIGS. 10-12) to the unlocked configuration (e.g., FIGS. 13-15) such that only one of the first button structure 250 and the second button structure 260 cannot shift the locking portion 230 and/or the locking mechanism 240 from the locked configuration (e.g., FIGS. 10-12) to the unlocked configuration (e.g., FIGS. 13-15) on its own. As such, the first button structure 250 and the second button structure 260 must be translated radially inward toward the central longitudinal axis of the locking portion 230 and/or the polymeric tube 238 by the user simultaneously and/or at the same time in order to shift the locking portion 230 and/or the locking mechanism 240 from the locked configuration (e.g., FIGS. 10-12) to the unlocked configuration (e.g., FIGS. 13-15).

It will be appreciated that a reversal of the elements shown in FIGS. 16-17 (e.g., the second button structure 260 is translated toward the central longitudinal axis of the locking portion 230 and/or the polymeric tube 238 by the user, and the first button structure 250 is not translated toward the central longitudinal axis of the locking portion 230 and/or the polymeric tube 238 by the user) is also possible and will produce substantially the same result.

After advancing the catheter 10 (e.g., the percutaneous circulatory support device) through the repositioning sheath 100/200 and positioning the blood pump 50 at a target site (e.g., within the heart of the patient), axial fixation of the catheter 10 (e.g., the percutaneous circulatory support device) may be desirable to ensure that the blood pump 50 is maintained in a desired position and/or in a proper position during use. In some situations, it may also be desirable and/or beneficial for medical personnel to reposition the catheter 10 (e.g., the percutaneous circulatory support device) and/or the blood pump 50 after insertion and/or during use. The locking portion 130/230 and/or the locking mechanism 140/240 may permit selective fixation of the catheter 10 (e.g., the percutaneous circulatory support device) relative to the repositioning sheath 100/200.

In some embodiments, the locking mechanism 140/240 may be configured to prevent relative axial translation of the elongate shaft 12 of the catheter 10 (e.g., the percutaneous circulatory support device) within and/or through the repositioning sheath 100/200, the locking portion 130/230, the lumen of the polymeric tube 138/238, and/or the locking mechanism 140/240. In at least some embodiments, the locking mechanism 140/240 may be configured to frictionally engage the polymeric tube 138/238 against the elongate shaft 12 of the catheter 10 (e.g., the percutaneous circulatory support device) disposed within the lumen of the polymeric tube 138/238 in the locked configuration.

In some embodiments, the locking mechanism 140/240 may be configured to deflect at least a portion of a wall of the polymeric tube 138/238 toward the central longitudinal axis of the polymeric tube 138/238 in the locked configuration, and the locking mechanism 140/240 may be configured to not deflect at least a portion of a wall of the polymeric tube 138/238 toward the central longitudinal axis of the polymeric tube 138/238 in the unlocked configuration. As such, the locking mechanism 140/240 may be configured to permit relative translation of the elongate shaft 12 of the catheter 10 (e.g., the percutaneous circulatory support device) through the lumen of the polymeric tube 138/238 in the unlocked configuration, and the locking mechanism 140/240 may be configured to prevent relative translation of the elongate shaft 12 of the catheter 10 (e.g., the percutaneous circulatory support device) through the lumen of the polymeric tube 138/238 in the locked configuration.

The repositioning sheath 100/200 described herein, when used with and/or in a percutaneous circulatory support system, may provide easy and intuitive operation, as well as quick and easy assembly. The repositioning sheath 100/200 described herein, when used with and/or in a percutaneous circulatory support system, may permit locking and unlocking of the position of the elongate shaft 12 of the catheter 10 (e.g., the percutaneous circulatory support device) relative to the repositioning sheath 100/200 without damaging the elongate shaft 12 of the catheter 10 (e.g., the percutaneous circulatory support device). The repositioning sheath 100/200 described herein, when used with and/or in a percutaneous circulatory support system, may allow for repositioning of the catheter 10 (e.g., the percutaneous circulatory support device) and/or the percutaneous circulatory support system both inside and outside the sterile field. The repositioning sheath 100/200 described herein, when used with and/or in a percutaneous circulatory support system, may enhance procedural flexibility and maintain the sterile field around the portion of the catheter 10 (e.g., the percutaneous circulatory support device) configured to engage with and/or access the vasculature of the patient. Other benefits and/or advantages are also contemplated.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the systems and/or devices described herein, as the discussion may be applied to other elements, members, components, or devices disclosed herein, and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, acrylonitrile butadiene styrene (ABS), epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP).

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler (e.g., barium sulfate, bismuth trioxide, bismuth oxychloride, or bismuth subcarbonate), and the like. Additionally, other radiopaque marker bands and/or coils may also be incorporated into the design of the system to achieve the same result.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment in other embodiments. The scope of the disclosure is, of course, defined in the language in which the appended claims are expressed.

What is claimed is:

1. A repositioning sheath for use in a percutaneous circulatory support system, comprising:
a body portion including a lumen extending therethrough;
a sheath extending distally from the body portion, the sheath including a lumen in communication with the lumen of the body portion, wherein the sheath is configured to fluidly engage with a vasculature of the patient; and
a locking portion secured to the body portion, wherein the locking portion is configured to shift between an unlocked configuration and a locked configuration;
wherein the locking portion includes:
a plurality of beams extending longitudinally from the body portion;
a polymeric tube disposed radially inward of the plurality of beams, the polymeric tube including a lumen in communication with the lumen of the body portion; and
a locking mechanism configured to selectively engage the polymeric tube against an elongate shaft disposed within the lumen of the polymeric tube in the locked configuration.

2. The repositioning sheath of claim 1, wherein the locking mechanism includes:
at least one ramp element extending radially outward from each beam of the plurality of beams; and
a rotatable member disposed radially outward of the plurality of beams;
wherein the rotatable member is configured to engage the at least one ramp element extending radially outward from each beam of the plurality of beams to shift the locking portion between the unlocked configuration and the locked configuration.

3. The repositioning sheath of claim 2, wherein the plurality of beams are circumferentially spaced apart from each other.

4. The repositioning sheath of claim 1, wherein the locking portion is configured to prevent relative translation of the elongate shaft through the lumen of the polymeric tube in the locked configuration.

5. The repositioning sheath of claim 1, wherein the locking portion is configured to permit relative translation of the elongate shaft through the lumen of the polymeric tube in the unlocked configuration.

6. The repositioning sheath of claim 1, wherein the body portion includes a pointer configured to cooperate with the rotatable member to visually communicate whether the locking portion is in the unlocked configuration or the locked configuration.

7. The repositioning sheath of claim 6, wherein the rotatable member includes a locked indicator disposed on an outer surface of the rotatable member and an unlocked indicator disposed on the outer surface of the rotatable member.

8. The repositioning sheath of claim 1, wherein the locking portion includes at least one first stop element, wherein the at least one first stop element is configured to engage at least one second stop element formed in the rotatable member in the unlocked configuration and wherein the at least one first stop element is configured to engage at least one third stop element formed in the rotatable member in the locked configuration.

9. A percutaneous circulatory support system, comprising:
a blood pump;
a housing;
an elongate shaft extending from the blood pump to the housing; and
a repositioning sheath comprising:
a body portion including a lumen extending therethrough;
a sheath extending distally from the body portion, the sheath including a lumen in communication with the lumen of the body portion, wherein the sheath is configured to fluidly engage with a vasculature of the patient; and
a locking portion secured to the body portion, wherein the locking portion is configured to shift between an unlocked configuration and a locked configuration;
wherein a sterile sleeve extends from the housing and surrounds at least a portion of the elongate shaft;
wherein the sterile sleeve is configured to releasably couple to the locking portion of the repositioning sheath.

10. The percutaneous circulatory support system of claim 9, wherein the locking portion of the repositioning sheath includes a proximal attachment structure configured to releasably couple a distal hub of the sterile sleeve to the locking portion of the repositioning sheath.

11. The percutaneous circulatory support system of claim 10, wherein the proximal attachment structure includes a pair of tabs biased radially apart from each other.

12. The percutaneous circulatory support system of claim 11, wherein each tab of the pair of tabs comprises a ledge configured to engage with the distal hub of the sterile sleeve to couple the distal hub of the sterile sleeve to the locking portion of the repositioning sheath.

13. The percutaneous circulatory support system of claim 12, wherein the pair of tabs is configured to be squeezed radially toward each other to release the distal hub of the sterile sleeve from the locking portion of the repositioning sheath.

14. The percutaneous circulatory support system of claim 10, wherein the distal hub of the sterile sleeve is configured to rotate relative to the proximal attachment structure of the locking portion of the repositioning sheath.

15. The percutaneous circulatory support system of claim 9, wherein the locking portion of the repositioning sheath includes:
a plurality of beams extending longitudinally from the body portion;
a polymeric tube disposed radially inward of the plurality of beams, the polymeric tube including a lumen in communication with the lumen of the body portion; and
a locking mechanism configured to selectively engage the polymeric tube against the elongate shaft when the elongate shaft is disposed within the lumen of the polymeric tube in the locked configuration.

16. The percutaneous circulatory support system of claim 15, wherein the proximal attachment structure is fixedly attached to the plurality of beams.

17. The percutaneous circulatory support system of claim 15, wherein the locking mechanism is configured to permit the elongate shaft to slide therethrough when the elongate shaft is disposed therein in the unlocked configuration and the locking mechanism is configured to prevent the elongate shaft from sliding therethrough when the elongate shaft is disposed therein in the locked configuration.

18. A repositioning sheath for use in a percutaneous circulatory support system, comprising:
a body portion including a lumen extending therethrough;
a sheath extending distally from the body portion, the sheath including a lumen in communication with the lumen of the body portion, wherein the sheath is configured to fluidly engage with a vasculature of the patient; and
a locking portion secured to the body portion, wherein the locking portion is configured to shift between an unlocked configuration and a locked configuration;
wherein the locking portion includes:
a polymeric tube including a lumen in communication with the lumen of the body portion, wherein the lumen of the polymeric tube is configured to receive an elongate shaft therein; and
a locking mechanism configured to squeeze the polymeric tube against the elongate shaft when the elongate shaft is disposed within the lumen of the polymeric tube in the locked configuration;
wherein the locking portion is self-biased toward the locked configuration.

19. The repositioning sheath of claim 18, wherein the locking mechanism includes:
a first button structure;
a second button structure disposed opposite the first button relative to a central longitudinal axis of the polymeric tube, wherein the second button structure is slidably engaged with the first button structure;
a first spring configured to bias a user-engageable portion of the first button structure away from the central longitudinal axis of the polymeric tube; and
a second spring configured to bias a user-engageable portion of the second button structure away from the central longitudinal axis of the polymeric tube.

20. The repositioning sheath of claim 19, wherein the first button structure and the second button structure cooperate to shift the locking portion from the locked configuration to the unlocked configuration such that only one of the first button structure and the second button structure cannot shift the locking portion from the locked configuration to the unlocked configuration on its own.
